(19) United States
(12) Patent Application Publication
(10) Pub. No.: US 2023/0051239 A1
(43) Pub. Date: Feb. 16, 2023

(54) COMPOSITIONS OF HYDROGELS AND METHODS OF USE THEREOF

(71) Applicant: Repertoire Immune Medicines, Inc., Cambridge, MA (US)

(72) Inventors: Assaf Rotem, Newton, MA (US); Ellen Lovisa Larsdotter Afzelius, Belmont, MA (US); Gang Hu, Newton, MA (US); Joanna Swain, Concord, MA (US); Ohad Yosefson, Concord, MA (US); Benjamin Peter Roscoe, Somerville, MA (US); Peter Lyubomirov Rogov, Winchester, MA (US); Harsh Jayeshkumar Vaidya, Somerville, MA (US)

(21) Appl. No.: 17/790,692

(22) PCT Filed: Dec. 31, 2020
(86) PCT No.: PCT/US2020/067708

§ 371 Date: Jul. 1, 2022

Related U.S. Application Data
(60) Provisional application No. 62/957,015, filed on Jan. 3, 2020.

(30) Foreign Application Priority Data
Jan. 3, 2020 (US) PCT/US2020/012236

(51) Int. Cl. C12P 21/06; C12P 21/02; C08F 220/56; C08J 9/00; C08J 3/075

(52) U.S. Cl. CPC C12P 21/06; C12P 21/02; C08F 220/56; C08J 9/0033; C08J 3/075; C08J 2333/26

(57) ABSTRACT

Described herein are compositions of hydrogels and methods of use thereof.

### CROSS-REFERENCE

This application claims the benefit of U.S. Provisional Patent Application No. 62/957,015, filed Jan. 3, 2020, and is a continuation-in-part of International Application PCT/US20/12236, filed Jan. 3, 2020, each of which is entirely incorporated herein by reference.

### BACKGROUND OF THE INVENTION

Conventional hydrogels formed with crosslinking have pore sizes that may not be compatible with methods, such as amplification, involving large molecules.

### SUMMARY OF THE INVENTION

The present disclosure provides compositions of hydrogels, methods of forming hydrogels, solutions for forming hydrogels, and methods of using hydrogels. More specifically, the present disclosure relates to hydrogels formed with crosslinking that have pore sizes that may be compatible with uses such as amplification involving large molecules.

In an aspect, the present disclosure provides a hydrogel, the hydrogel comprising: (i) a polymer structure, wherein the polymer structure comprises a plurality of monomer units and at least one of the monomer units is a cross-linking unit; and (ii) a plurality of pores in the polymer structure, the plurality of pores having an average pore size from 0.1 μm to 60 μm.

In some embodiments, the pore size is from 1 μm to 50 μm. In some embodiments, the plurality of monomer units is from 0.1 wt % to 90 wt % of the hydrogel. In some embodiments, the plurality of monomer units is from 2.75 wt % to 6 wt % of the hydrogel. In some embodiments, from 0.1% to 90% of the monomer units are cross-linking units. In some embodiments, from 18% to 35% of the monomer units are cross-linking units. In some embodiments, at least one linker is attached to at least one of the plurality of monomer units. In some embodiments, the polymer structure further comprises a target molecule attached to at least one linker. In some embodiments, the linker comprises a nucleic acid. In some embodiments, the linker comprises an oligonucleotide. In some embodiments, the linker is covalently attached to the at least one monomer unit. In some embodiments, the linker further comprises a protein binding domain. In some embodiments, the target molecule comprises a protein, a nucleic acid molecule, a peptide, a biomolecule, a drug, a chemical moiety, a lipid, any derivative thereof, or any combination thereof. In some embodiments, the target molecule is a nucleic acid molecule, and wherein the target molecule is covalently attached to the linker. In some embodiments, the target molecule is a multimeric protein. In some embodiments, the target molecule is a heteromultimer. In some embodiments, the multimeric protein is produced by in vitro transcription/translation (IVTT). In some embodiments, the multimeric protein is produced in mammalian cells or bacterial cells. In some embodiments, the target molecule is attached to the protein binding domain. In some embodiments, the polymer structure further comprises: a first nucleic acid molecule attached to at least one of the plurality of monomer units, the first nucleic acid molecule comprising a first sequence; and a second nucleic acid molecule attached to at least a one of the plurality of monomer units, the second nucleic acid molecule comprising a second sequence. In some embodiments, the first sequence encodes a cleavage moiety. In some embodiments, the first sequence encodes an endoprotease cleavage moiety. In some embodiments, the second sequence encodes at least a portion of a polypeptide. In some embodiments, the hydrogel has a diameter less than 250 μm. In some embodiments, the plurality of monomer units comprise a PEG monomer, an acrylamide monomer, a PEGDA monomer, a chitosan, an alginate, a gelatin, a hyaluronic acid, a chondroitin, a fibrinogen, a peptide, a polyfumerate, a phosphoester, any derivative thereof, or any combination thereof. In some embodiments, the hydrogel further comprises a fluorophore. In some embodiments, the plurality of pores is located at the exterior surface of the polymer structure. In some embodiments, the plurality of pores is embedded within the polymer structure. In some embodiments, at least a portion of the hydrogel can be enzymatically degraded. In some embodiments, at least a portion of the hydrogel can be hydrolytically degraded.

In another aspect, the present disclosure provides a solution for producing a hydrogel, the solution comprising: (i) from 0.1 wt % to 90 wt % monomer units, wherein from 0.1% to 90% of the monomer units are cross-linking units; (ii) a radical initiator; and (iii) a solvent. In some embodiments, the solution comprises from 2.75 wt % to 6 wt % monomer units. In some embodiments, from 18% to 35% of the monomer units are cross-linking units. In some embodiments, the solution further comprises a surfactant. In some embodiments, the solvent comprises water.

In yet another aspect, the present disclosure provides a method of making a hydrogel, the method comprising: (i) obtaining a solution, the solution comprising from 0.1 wt % to 90 wt % monomer units wherein from 0.1% to 90% of the monomer units are cross-linking units, a radical initiator, and a solvent; (ii) using the radical initiator to polymerize the monomer units; and (iii) curing the polymer structure. In some embodiments, the solution comprises from 2.75 wt % to 6 wt % monomer units. In some embodiments, from 18% to 35% of the monomer units are cross-linking units. In some embodiments, the method further comprises the step of punching out polymer structure. In some embodiments, the solvent comprises water. In some embodiments, the solution further comprises a surfactant. In some embodiments, the solution is within a droplet. In some embodiments, the method further comprises generating a droplet comprising the solution. In some embodiments, the method further comprises curing the polymer structure within the droplet.

In yet another aspect, the present disclosure provides a method of producing a target molecule complex using a hydrogel as disclosed herein, the method comprising: (i) providing the hydrogel, wherein the hydrogel comprises a nucleic acid molecule, and the nucleic acid molecule comprises a cleavage moiety; (ii) transcribing and translating the nucleic acid molecule to produce a plurality of target molecules; and (iii) cleaving the cleavage moiety, thereby producing the target molecule complex.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts hydrogels having 6 wt % monomer, 3% of which is a cross-linking monomer, in a study assessing the diffusion of fluorescein isothiocyanate (FITC)-labeled dextran.

FIG. 1B depicts hydrogels having 3.75 wt % monomer, 20% of which is a cross-linking monomer, in a study assessing the diffusion of FITC-labeled dextran.

FIG. 1C depicts hydrogels having 3.5 wt % monomer, 24.3% of which is a cross-linking monomer, in a study assessing the diffusion of FITC-labeled dextran.

FIG. 2A depicts hydrogels having 6 wt % monomer, 3% of which is a cross-linking monomer, as observed under an optical microscope.

FIG. 2B depicts hydrogels having 3.75 wt % monomer, 20% of which is a cross-linking monomer, as observed under an optical microscope.

FIG. 3 depicts a 696-fold magnification of hydrogels having 6 wt % monomer, 3% of which is a cross-linking monomer, as observed under a Scanning Electron Microscope (SEM).

FIG. 4 depicts 10,400-fold magnification of hydrogels having 6 wt % monomer, 3% of which is a cross-linking monomer, as observed under SEM

FIG. 5 depicts 1,220-fold magnification of hydrogels having 3.75 wt % monomer, 20% of which is a cross-linking monomer, as observed under SEM.

FIG. 6 depicts 7,450-fold magnification of hydrogels having 3.75 wt % monomer, 20% of which is a cross-linking monomer, as observed under SEM.

FIG. 7A depicts confocal imagery of hydrogels modified to bind to multimeric protein attachments.

FIG. 7B depicts confocal imagery of hydrogels not modified to bind to multimeric protein attachments.

FIG. 8A depicts confocal imagery of hydrogels attached to a target moiety via a cleavable linker.

FIG. 8B depicts confocal imagery of hydrogels following enzymatic cleavage of the cleavable linker.

FIG. 8C depicts confocal imagery of hydrogels that were not exposed to the enzymatic cleavage, and retain the linker.

FIG. 9 depicts the Western analysis of cleaved molecules that had previously been attached to a hydrogel via a cleavable linker.

FIG. 10 depicts a schematic of a nucleic acid construct that can be used in compositions and methods of the disclosure. The construct can encode a peptide and an identifier (e.g., a self-identifier that corresponds to all or a part of the coding sequence of the peptide). The locations of the forward and reverse primers are indicated (e.g., primers that can be used in Examples 5-7).

FIG. 11A and FIG. 11B depict PCR amplification of full length antigen-encoding templates (FIG. 11A) and identifiers (FIG. 11B) onto hydrogels as outlined in Examples 6 and 7.

FIGS. 12A-12C depict the generation of folded and identifier-tagged sc-pMHC. FIG. 12A provides microscopy images that demonstrate a sc-pMHC of the disclosure was in vitro transcribed and translated as outlined in Example 8. FIG. 12B provides ELISA results that demonstrate release of folded sc-pMHC multimers as outlined in Example 9. FIG. 12C provides a Western Blot that demonstrates the sc-pMHC is tagged with an identifier as outlined in Example 9.

### DETAILED DESCRIPTION OF THE INVENTION

### Introduction

The compositions, solutions, and methods of using hydrogels disclosed herein solve significant technical problems related to efficiently using hydrogels with large molecules. For example, for using hydrogels with large molecules in amplification reactions. This is especially relevant to the amplification or targeting of large target molecules, such as long nucleic acid molecules or multimeric proteins. Hydrogels produced in conventional methods have pores that may be too small to release amplified products — that is, an amplification target may enter a pore, but following amplification cannot exit. Previous syntheses of hydrogels found that increasing the concentration of crosslinking units in the hydrogel led to decreased porosity and smaller pore sizes (see, e.g.: Chavda et al. INT. J. PHARM. INVESTIG. 2011 January-March; 1(1):17-21).

Disclosed herein is the surprising discovery that increasing the concentration of crosslinking units in some hydrogel compositions can actually increase pore sizes, and thus increase porosity. The increased pore size allows for increased diffusion of compounds in solution, and decreases the likelihood of trapping large target molecules, for example, after amplification or after linking amplified products. Further disclosed herein are methods of producing said hydrogel compositions, and methods of using the same.

### Definitions

As used herein, the term “cleavage moiety” refers to a motif or sequence that is cleavable. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc. In some embodiments, the cleavage moiety comprises a nucleotide. In certain embodiments, the cleavage moiety comprising a nucleotide can be cleaved by a restriction enzyme.

As used herein, the term “cross-linking unit” can refer to a molecule that links to another (same or different) molecule. In some embodiments, the cross-linking unit is a monomer. In some embodiments, the cross-link is a chemical bond. In some embodiments, the cross-link is a covalent bond. In some embodiments, the cross-link is an ionic bond. In some embodiments, the cross-link alters at least one physical property of the linked molecules, e.g., a polymer's physical property.

As used herein, the term “hydrogel” can refer to a cross-linked network of monomers. In some embodiments, the hydrogel comprises a polymer structure with a plurality of monomer units. In some embodiments, at least one of the monomer units is a cross-linking unit.

As used herein, the term “monomer” can refer to a molecule that makes up at least a portion of the polymer structure. In some embodiments, monomers can be a molecular building block of the polymer structure. In some embodiments, monomers can undergo polymerization to form a polymer structure. Monomers (also referred to herein as monomer units or monomeric units) can be crosslinking (i.e., have multiple functional groups and can form chemical links between molecular chains), or can be non-crosslinking. As a non-limiting example, a crosslinking monomer can be bis-acrylamide, while a non-crosslinking monomer can be acrylamide. A plurality of monomers can attach to one another in order to form a polymer chain, a branched polymer, a crosslinked polymer, and the like. As a non-limiting example, acrylamide monomers can undergo polymerization to form polyacrylamide, while bis-acrylamide can be added to the polymerization to form a cross-linked polymer structure.

In some embodiments, the mass of total monomers present in a hydrogel can be represented by %T. As used herein, the term %T refers to the total sum of monomers (both crosslinking monomers and non-crosslinking monomers) divided by the total mass of the hydrogel, as provided by equation 1, below. Alternatively, %T can be calculated as the total mass of monomers per 100 mL of non-swelled hydrogel.

$$\% T = \frac{\begin{matrix}mass of non ‐ crosslinking monomers + \\ mass of crosslinking unit ( s )\end{matrix}}{total mass of hydrogel} \qquad ( eq . 1 )$$

In some embodiments, the mass percent of crosslinking monomers present in a hydrogel can be represented by %C. As used herein, the term %C refers to the total percent mass of the crosslinking unit(s) present in the hydrogel, divided by the total mass of all monomers, as provided by equation 2, below.

$$\% C = \frac{mass of crosslinking unit ( s )}{\begin{matrix}mass of non ‐ crosslinking monomers + \\ mass of crosslinking unit ( s )\end{matrix}} \qquad ( eq . 2 )$$

As used herein, the term “polymer structure” can refer to an ordered structure of molecules.

As used herein, the term “pore” or “pores” can refer to a space or spaces within a polymer structure.

As used herein, the term “target molecule” can refer to a moiety that can be associated or attached to the hydrogel or monomer. In some embodiments, the target molecule comprises a protein, a nucleic acid molecule, a peptide, a biomolecule, a drug, a chemical moiety, a lipid, a cell, any derivative thereof, or any combination thereof.

### Hydrogel Composition

Disclosed herein is a hydrogel comprising a polymer structure, wherein the polymer structure comprises a plurality of monomer units, and at least one of the monomer units is a cross-linking unit. The hydrogel further comprises a plurality of pores in the polymer structure. As disclosed herein, large pore sizes can be favorable to amplification reactions or linking of target molecules taking place at the surface of, and/or in proximity to, the hydrogel.

In some embodiments, the plurality of pores have an average pore size of less than 300 p.m in radius, less than 200 μm in radius, less than 150 μm in radius, less than 100 μm in radius, less than 90 μm in radius, less than 80 μm in radius, less than 70 μm in radius, less than 60 μm in radius, less than 50 μm in radius, less than 40 μm in radius, less than 30 μm in radius, less than 20 μm in radius, less than 10 μm in radius, less than 1 μm in radius, less than 900 nm in radius, less than 800 nm in radius, less than 700 nm in radius, less than 600 nm in radius, less than 500 nm in radius, less than 400 nm in radius, less than 300 nm in radius, less than 200 nm in radius, less than 100 nm in radius, or 1 nm in radius. In some embodiments, the plurality of pores have an average pore size of greater than 1 nm in radius, 10 nm in radius, greater than 20 nm in radius, greater than 30 nm in radius, greater than 40 nm in radius, greater than 50 nm in radius, greater than 60 nm in radius, greater than 70 nm in radius, greater than 80 nm in radius, greater than 90 nm in radius, greater than 100 nm in radius, greater than 200 nm in radius, greater than 300 nm in radius, greater than 400 nm in radius, greater than 500 nm in radius, greater than 600 nm in radius, greater than 700 nm in radius, greater than 800 nm in radius, greater than 900 nm in radius, greater than 1 μm in radius, great than 5 μm in radius, greater than 10 μm in radius, greater than 15 μm in radius, greater than 20 μm in radius, greater than 25 μm in radius, greater than 30 μm in radius, greater than 35 μm in radius, greater than 40 μm in radius, greater than 45 p.m in radius, greater than 50 μm in radius, greater than 55 μm in radius, or greater than 60 μm in radius. In some embodiments, the plurality of pores have an average pore size of less than 300 μm in diameter, less than 200 μm in diameter, less than 150 μm in diameter, less than 100 μm in diameter, less than 90 μm in diameter, less than 80 μm in diameter, less than 70 μm in diameter, less than 60 μm in diameter, less than 50 μm in diameter, less than 40 μm in diameter, less than 30 μm in diameter, less than 20 μm in diameter, less than 10 μm in diameter, less than 1 μm in diameter, less than 900 nm in diameter, less than 800 nm in diameter, less than 700 nm in diameter, less than 600 nm in diameter, less than 500 nm in diameter, less than 400 nm in diameter, less than 300 nm in diameter, less than 200 nm in diameter, less than 100 nm in diameter, or 1 nm in diameter. In some embodiments, the plurality of pores have an average pore size of greater than 1 nm in diameter, 10 nm in diameter, greater than 20 nm in diameter, greater than 30 nm in diameter, greater than 40 nm in diameter, greater than 50 nm in diameter, greater than 60 nm in diameter, greater than 70 nm in diameter, greater than 80 nm in diameter, greater than 90 nm in diameter, greater than 100 nm in diameter, greater than 200 nm in diameter, greater than 300 nm in diameter, greater than 400 nm in diameter, greater than 500 nm in diameter, greater than 600 nm in diameter, greater than 700 nm in diameter, greater than 800 nm in diameter, greater than 900 nm in diameter, greater than 1 μm in diameter, great than 5 μm in diameter, greater than 10 μm in diameter, greater than 15 μm in diameter, greater than 20 μm in diameter, greater than 25 μm in diameter, greater than 30 μm in diameter, greater than 35 μm in diameter, greater than 40 μm in diameter, greater than 45 μm in diameter, greater than 50 μm in diameter, greater than 55 μm in diameter, or greater than 60 μm in diameter.

In some embodiments, the plurality of pores have an average pore size from 1 nm to 200 μm in radius, 10 nm to 200 μm in radius, 100 nm to 200 μm in radius, 200 nm to 200 μm in radius, 10 nm to 60 μm in radius, from 500 nm to 150 μm in radius, from 600 nm to 140 μm in radius, from 700 nm to 130 μm in radius, from 800 nm to 120 μm in radius, from 900 nm to 110 μm in radius, from 1 μm to 100 μm in radius, from 1 μm to 90 μm in radius, from 1 μm to 80 μm in radius, from 1 μm to 70 μm in radius, from 1 μm to 60 μm in radius, from 1 μm to 50 μm in radius, from 1 μm to 40 μm in radius, or from 1 μm to 30 μm in radius. In some embodiments, the plurality of pores have an average pore size from 1 nm to 200 μm in diameter, 10 nm to 200 μm in diameter, 100 nm to 200 μm in diameter, 200 nm to 200 μm in diameter, 10 nm to 60 μm in diameter, from 500 nm to 150 μm in diameter, from 600 nm to 140 μm in diameter, from 700 nm to 130 μm in diameter, from 800 nm to 120 μm in diameter, from 900 nm to 110 μm in diameter, from 1 μm to 100 μm in diameter, from 1 μm to 90 μm in diameter, from 1 μm to 80 μm in diameter, from 1 μm to 70 μm in diameter, from 1 μm to 60 μm in diameter, from 1 μm to 50 μm in diameter, from 1 μm to 40 μm in diameter, or from 1 μm to 30 μm in diameter.

In some embodiments, the average pore size of the hydrogel is from 0.25 μm to 2.5 μm, from 0.25 μm to 3 μm, from 0.25 μm to 5 μm, from 0.5 μm to 5 pm, from 0.5 μm to 6 μm, from 0.5 μm to 8 μm, from 0.5 μm to 10 μm, from 1 μm to 5 μm, from 1 μm to 6 μm, from 1 μm to 8 μm, or from 1 μm to 10 μm in radius. In some embodiments, the average pore size of the hydrogel is from 0.25 μm to 2.5 μm, from 0.25 μm to 3 μm, from 0.25 μm to 5 μm, from 0.5 μm to 5 pm, from 0.5 μm to 6 μm, from 0.5 μm to 8 μm, from 0.5 μm to 10 μm, from 1 μm to 5 μm, from 1 μm to 6 μm, from 1 μm to 8 μm, or from 1 μm to 10 μm in diameter.

In some embodiments, the average size of the plurality of pores corresponds with the monomer composition of the polymer structure of the hydrogel. The polymer structure comprises a plurality of monomer units, wherein some of the monomer units are non-crosslinking monomers (i.e., they are not crosslinking units), and some of the monomer units are crosslinking units. As disclosed in detail above, in some cases, the combined mass of these monomers divided by the total mass of the hydrogel is referred to as the %T of the hydrogel. In some embodiments, the %T of the hydrogel is no more than 90 wt %, no more than 80 wt %, no more than 70 wt %, no more than 60 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 9 wt %, no more than 8 wt %, no more than 7 wt %, no more no more 6 wt %, no more than wt %, no more than 4 wt %, no more than 3 wt %, no more than 2 wt %, or no more than 1 wt %. In some embodiments, the %T of the hydrogel is from 0.5 wt % to 20 wt %, from 1 wt % to 15 wt %, from 1.5 wt % to 15 wt %, from 1.75 wt % to 12.5 wt %, from 2 wt % to 10 wt %, from 2.5 wt % to 8 wt %, from 3 wt % to 6 wt %, from 0.1 wt % to 90 wt %, from 0.1 wt % to 80 wt %, from 0.1 wt % to 70 wt %, from 0.1 wt % to 60 wt %, from 0.1 wt % to 50 wt %, from 0.1 wt % to 40 wt %, from 0.1 wt % to 30 wt %, from 0.1 wt % to 20 wt %, from 0.1 wt % to 10 wt %, from 0.1 wt % to 1 wt %, from 1 wt % to 90 wt %, from 1 wt % to 80 wt %, from 1 wt % to 70 wt %, from 1 wt % to 60 wt %, from 1 wt % to 50 wt %, from 1 wt % to 40 wt %, from 1 wt % to 30 wt %, from 1 wt % to 20 wt %, from 1 wt % to 10 wt %, or from 1 wt % to 5 wt %. In certain embodiments, the %T of the hydrogel is from 2.75 wt % to 6 wt %. In certain embodiments, the %T of the hydrogel is from 3 wt % to 6 wt %.

In some cases, the combined mass of these monomers per 100 mL of non-swelled hydrogels is referred to as the %T of the hydrogel. In some embodiments, the %T of the hydrogel is no more than 90% w/v, no more than 80% w/v, no more than 70% w/v, no more than 60% w/v, no more than 50% w/v, no more than 45% w/v, no more than 40% w/v, no more than 35% w/v, no more than 30% w/v, no more than 25% w/v, no more than 20% w/v, no more than 15% w/v, no more than 10% w/v, no more than 9% w/v, no more than 8% w/v, no more than 7% w/v, no more no more 6% w/v, no more than % w/v, no more than 4% w/v, no more than 3% w/v, no more than 2% w/v, or no more than 1% w/v. In some embodiments, the %T of the hydrogel is from 0.5% w/v to 20% w/v, from 1% w/v to 15% w/v, from 1.5% w/v to 15% w/v, from 1.75% w/v to 12.5% w/v, from 2% w/v to 10% w/v, from 2.5% w/v to 8% w/v, from 3% w/v to 6% w/v, from 0.1% w/v to 90% w/v, from 0.1% w/v to 80% w/v, from 0.1% w/v to 70% w/v, from 0.1% w/v to 60% w/v, from 0.1% w/v to 50% w/v, from 0.1% w/v to 40% w/v, from 0.1% w/v to 30% w/v, from 0.1% w/v to 20% w/v, from 0.1% w/v to 10% w/v, from 0.1% w/v to 1% w/v, from 1% w/v to 90% w/v, from 1% w/v to 80% w/v, from 1% w/v to 70% w/v, from 1% w/v to 60% w/v, from 1% w/v to 50% w/v, from 1% w/v to 40% w/v, from 1% w/v to 30% w/v, from 1% w/v to 20% w/v, from 1% w/v to 10% w/v, or from 1% w/v to 5% w/v. In certain embodiments, the %T of the hydrogel is from 2.75% w/v to 6% w/v. In certain embodiments, the %T of the hydrogel is from 3% w/v to 6% w/v.

In some embodiments, the concentration of the monomer units or %T of the hydrogel is from 1.0 wt % to 3.0 wt %, from 2.0 wt % to 4.0 wt %, from 2.0 wt % to 5.0 wt %, from 3.0 wt % to 4.0 wt %, or from 3.0 wt % to 5.0 wt % in a non-swelled form. In some embodiments, the %T of the hydrogel is at least about 1.0 wt %, 2.0 wt %, 3.0 wt %, 4.0 wt %, 5.0 wt % or more in a non-swelled form. In some embodiments, the concentration of the monomer units or %T of the hydrogel is from 1.0% w/v to 3.0% w/v, from 2.0% w/v to 4.0% w/v, from 2.0% w/v to 5.0% w/v, from 3.0% w/v to 4.0% w/v, or from 3.0% w/v to 5.0% w/v in a non-swelled form. In some embodiments, the %T of the hydrogel is at least about 1.0% w/v, 2.0% w/v, 3.0% w/v, 4.0% w/v, 5.0% w/v or more in a non-swelled form.

As described further above, the mass of the crosslinking units divided by the total mass of the monomers (both the crosslinking units and non-crosslinking monomers) is referred to as the %C of the hydrogel. As disclosed herein, it has been surprisingly and unexpectedly discovered that, as provided herein, a large %C can increase pore size of the hydrogel. In some embodiments, the %C of the hydrogel is no less than 0.1%, no less than 1%, no less than 5%, no less than 10%, no less than 15%, no less than 16%, no less than 17%, no less than 18%, no less than 19%, no less than 20%, no less than 21%, no less than 22%, no less than 23%, no less than 24%, no less than 25%, no less than 26%, no less than 27%, no less than 28%, no less than 29%, no less than 30%, no less than 31%, no less than 32%, no less than 33%, no less than 34%, no less than 35%, no less than 36%, no less than 37%, no less than 38%, no less than 39%, no less than 40%, no less than 50%, no less than 60%, no less than 70%, no less than 80%, or no less than 90%. In some embodiments, the %C of the hydrogel is from 0.1% to 90%, from 0.1% to 80%, from 0.1% to 70%, from 0.1% to 60%, from 0.1% to 50%, from 0.1% to 40%, from 0.1% to 30%, from 0.1% to 20%, from 0.1% to 10%, from 0.1% to 5%, or from 0.1% to 1%. In some embodiments, the %C of the hydrogel is from 1% to 90%, from 1% to 80%, from 1% to 70%, from 1% to 60%, from 1% to 50%, from 1% to 40%, from 1% to 30%, from 1% to 20%, from 1% to 10%, or from 1% to 5%. In some embodiments, the %C of the hydrogel is from 15% to 40%, from 16% to 39%, from 17% to 38%, from 18% to 37%, from 18% to 36%, from 18% to 35%, from 19% to 34%, from 19% to 33%, from 19% to 32%, from 19% to 31%, from 19% to 30%, from 20% to 29%, from 20% to 28%, from 20% to 27%, from 20% to 26%, or from 20% to 25%. In some embodiments, the %C of the hydrogel is from 15% to 20%, from 20% to 25%, from 25% to 30%, or from 30% to 35%. In some embodiment, the %C of the hydrogel is from 15% to 16%, from 16% to 17%, from 17% to 18%, from 18% to 19%, from 19% to 20%, from 20% to 21%, from 21% to 22%, from 22% to 23%, from 23% to 24%, from 24% to 25%, from 25% to 26%, from 26% to 27%, from 27% to 28%, from 28% to 29%, from 29% to 30%, from 30% to 31%, from 31% to 32%, from 32% to 33%, from 33% to 34%, or from 34% to 35%. In certain embodiments, the %C of the hydrogel is from 18% to 35%. In certain embodiments, the %C of the hydrogel is from 18% to 25%.

In some embodiments, the concentration of the crosslinking units in the hydrogel is from 10 wt % to 20 wt %, from 10 wt % to 30 wt %, from 10 wt % to 40 wt %, from 10 wt % to 50 wt %, from 10 wt % to 60 wt %, from 20 wt % to 30 wt %, from 20 wt % to 40 wt %, from 20 wt % to 50 wt %, or from 20 wt % to 60 wt % of the total monomer units. In some embodiments, the concentration of the crosslinking units in the hydrogel is at least about 5 wt %, 10 wt %, 20 wt %, 30 wt % or more of the total monomer units. In some embodiments, the concentration of the crosslinking units in the hydrogel is at most about 60 wt %, 50 wt %, 40 wt %, 30 wt %, 20 wt % or less of the total monomer units. In some embodiments, the concentration of the crosslinking units in the hydrogel is from 10% w/v to 20% w/v, from 10% w/v to 30% w/v, from 10% w/v to 40% w/v, from 10% w/v to 50% w/v, from 10% w/v to 60% w/v, from 20% w/v to 30% w/v, from 20% w/v to 40% w/v, from 20% w/v to 50% w/v, or from 20% w/v to 60% w/v of the total monomer units. In some embodiments, the concentration of the crosslinking units in the hydrogel is at least about 5% w/v, 10% w/v, 20% w/v, 30% w/v or more of the total monomer units. In some embodiments, the concentration of the crosslinking units in the hydrogel is at most about 60% w/v, 50% w/v, 40% w/v, 30% w/v, 20% w/v or less of the total monomer units.

In some embodiments, the hydrogel comprises monomers that are naturally derived. In certain embodiments, the monomer can comprise a dextran, a gelatin, styrenated gelatin, a thiol-modified gelatin, a chitosan, chitosan-g-azidobenzoic acid, a hyaluronic acid, acrylated hyaluronic acid, methacrylated hyaluronic acid, thiol-modified hyaluronic acid, a pectin, a lactic acid, an alginate, alginate dialdehyde, a chondroitin sulfate, methacrylated chondroitin sulfate, thiol-modified chondroitin sulfate, a fibrinogen, a PEGylated fibrinogen, fibrinogen-g-PEGacryloyl, a peptide, a carbohydrate, a sucrose, an elastin-like polypeptide (ELP), a genetically engineered ELP, a polyfumarate, poly(lactide-co-ethylene oxide-co-fumarate), MMP-diacrylate, a phosphoester, poly(t-aminohexyl propylene phosphate)-acrylate, any oligomer thereof, any derivative thereof, or any combination thereof.

In some embodiments, the hydrogel comprises monomers that are synthetically created or synthetically derived. In some embodiments, the monomer can comprise a polyethylene glycol (PEG), a polyethylene glycol diacrylate, a PEG thiol, acryloyl-PEG-RGD, PCL-b-PEG-b-PCL dimethacrylate, a polyglycerol succinic acid, PEG-(poly(glycerol succinic acid methacrylate))₂, acrylic acid, an acrylamide, a bisacrylamide, a polystyrene, a poly(styrenesulphonate), an alkylamine, an octylamine, a polyacrylate, a polyacrilic acid, a methacrylate, an oligo(polyethylene glycol) fumarate, N-vinylpyrrolidone, any derivative thereof, or any combination thereof.

In some embodiments, the plurality of monomer units comprises a PEG monomer, an acrylamide monomer, a PEGDA monomer, a chitosan, an alginate, a gelatin, a hyaluronic acid, a chondroitin, a fibrinogen, a peptide, a polyfumerate, a phosphoester, any derivative thereof, or any combination thereof.

In order to initiate or assist with polymerization, an initiator can be used. Any initiator appropriate for the polymerization can be used. In some embodiments, the initiator comprises: 2-hydroxy-2-methyl-1-phenyl-propan-1-one (Darocur 1173); 2,2-dimethoxy-2-phenylacetophenone (DMPA); 2-hydroxy-2-methylpropiophenone (HOMPP); ammonium persulfate (APS); tetramethylethylenediamene (TEMED); 1-[4-(2-hydroxyethoxy)-phenyl]-2-hydroxy-2-methyl-1-propane-1-one (Irgacure 2959); an organic salt; barium acetate; Borax; camphorquinone; an enzyme; tissue transglutaminase (optionally with calcium chloride); Eosin-Y; any derivative thereof; or any combination thereof.

In some embodiments, the initiator comprises a free radical initiator, an ATRP initiator, an NMP initiator, an ionic polymerization initiator, an amine photochemical coinitiator, an organic photoinitiator, any derivative thereof, or any combination thereof. In some embodiments, the free radical initiator comprises an azo compound, an inorganic peroxide, an organic peroxide, any derivative thereof, or any combination thereof. In some embodiments, the organic photoinitiator comprises an acetophenone, a benzil compound, an benzoin compound, a benzophenone, a cationic photoinitiator, a thioxanthone, anthraquinone-2-sulfonic acid, 2-tert-butylanthraquinone, camphorquinone, diphenyl(2,4,6-trimethylbenzoyl)phosphine oxide, lithium phenyl-2,4,6-trimethylbenzoylphosphinate, 9,10-phenanthrenequinone, phenylbis(2,4,6-trimethylbenzoyl)phosphine oxide, any derivative thereof, or any combination thereof.

The formation of the polymer structure involves a polymerization synthesis. In some embodiments, the polymerization synthesis comprises activation of polymerization by light. As non-limiting examples, the polymerization synthesis can comprise activation by ultraviolet (UV) light, for example, by light having a wavelength of 365 nm. In some embodiments, the polymerization synthesis comprises chemical activation. In some embodiments, the polymerization synthesis comprises biochemical activation. As a non-limiting example of biochemical activation, an enzyme can be added to a mixture of monomers in order to aid the polymerization and formation of a polymer structure.

In some embodiments, the polymer structure comprises an oligonucleotide, a methacrylated oligonucleotide, an acrylated oligonucleotide, a functionalized oligonucleotide, an acrylated antibody structure, a protein structure, a carbohydrate chain, an oligosaccharide, a plurality of synthetic polymer chains, any derivative thereof, or any combination thereof.

The hydrogels disclosed herein can be used in various settings, for example, in a laboratory setting. As a non-limiting example, an individual hydrogel can be encapsulated with a sample liquid in a droplet. It can be beneficial to use hydrogels having a small diameter. In some embodiments, the hydrogels have an average diameter less than 1000 μm, less than 900 μm, less than 800 μm, less than 700 μm, less than 600 μm, less than 500 μm, less than 400 μm, less than 300 μm, less than 200 μm, less than 100 μm, less than 90 μm, less than 80 μm, less than 70 μm, less than 60 μm, or less than 50 μm. In some embodiments, the hydrogels have an average diameter from 900 μm to 50 μm, from 800 μm to 60 μm, from 700 μm to 70 μm, from 600 μm to 80 μm, from 500 μm to 90 μm, or from 400 μm to 100 μm.

In some embodiments, the hydrogels disclosed herein can be used in a well. As a non-limiting example, an individual hydrogel can be encapsulated in a single well. In some embodiments, a plurality of wells comprise a plurality of hydrogels. In certain embodiments, the plurality of wells are located on a plate. In some embodiments, the plate has 4 wells. In some embodiments, the plate has 6 wells. In some embodiments, the plate has 8 wells. In some embodiments, the plate has 12 wells. In some embodiments, the plate has 24 wells. In some embodiments, the plate has 48 wells. In some embodiments, the plate has 96 wells. In some embodiments, the plate has 384 wells. In some embodiments, the plate has 1536 wells. In some embodiments, the plate has from 1 to 100 wells. In some embodiments, the plate has from 100 to 500 wells. In some embodiments, the plate has from 500 to 2500 wells. In some embodiments, the plate has from 2500 to 12,500 wells. In some embodiments, the plate has greater than 12,500 wells.

In some embodiments, the number of hydrogels in each well can be individually varied. In some embodiments, the plurality of wells each has one hydrogel, two hydrogels, three hydrogels, four hydrogels, five hydrogels, six hydrogels, seven hydrogels, eight hydrogels, nine hydrogels, ten hydrogels, more than 10 hydrogels, more than 15 hydrogels, more than 20 hydrogels, more than 25 hydrogels, more than 50 hydrogels, or more than 100 hydrogels in each well. In some embodiments, the plurality of wells has an average number of hydrogels. In some embodiments, the average number of hydrogels in the plurality of wells is from 0.01 to 2, from 0.1 to 1.9, from 0.2 to 1.8, from 0.3 to 1.7, from 0.4 to 1.6, from 0.5 to 1.5, from 0.6 to 1.4, from 0.7 to 1.3, from 0.8 to 1.2, or from 0.9 to 1.1. In some embodiments, the average number of hydrogels in the plurality of wells is about 1. In some embodiments, the average number of hydrogels in the plurality of wells is from 0.5 to 1.5, from 1.5 to 2.5, from 2.5 to 3.5, from 3.5 to 4.5, from 4.5 to 5.5, from 5.5 to 6.5, from 6.5 to 7.5, from 7.5 to 8.5, from 8.5 to 9.5, or from 9.5 to 10.5. In some embodiments, the average number of hydrogels in the plurality of wells is less than 100, less than 90, less than 80, less than 70, less than 60, less than 50, less than 40, less than 30, less than 20, less than 10, less than 9, less than 8, less than 7, less than 6, less than 5, less than 4, less than 3, less than 2, or less than 1.

In some embodiments, the hydrogels disclosed herein can be used in wells of assorted sizes. In some embodiments, the plurality of wells has an average volume of less than 10 mL, less than 5 mL, less than 4 mL, less than 3 mL, less than 2 mL, less than 1 mL, less than 900 μL, less than 800 μL, less than 700 μL, less than 600 μL, less than 500 μL, less than 400 μL, less than 300 μL, less than 200 μL, less than 150 μL, less than 125 μL, less than 120 μL, less than 110 μL, less than 100 μL, less than 90 μL, less than 80 μL, less than 70 μL, less than 60 μL, less than 50 μL, less than 40 μL, less than 30 μL, less than 20 μL, less than 10 μL, less than 5 μL, or less than 1 μL. In some embodiments, the plurality of wells has an average cross-sectional diameter of less than 2 cm, less than 1.5 cm, less than 1 cm, less than 9 mm, less than 8 mm, less than 7 mm, less than 6 mm, less than 5 mm, less than 4 mm, less than 3 mm, less than 2 mm, less than 1 mm, less than 900 less than 800 less than 700 less than 600 less than 500 less than 400 less than 300 less than 200 less than 100 less than 90 less than 80 less than 70 less than 60 less than 50 less than 40 less than 30 or less than 20 In some embodiments, the plurality of wells has an average cross-sectional diameter from 200 μm to 5 from 180 μm to 10 from 160 μm to 15 from 140 μm to 20 from 120 μm to 25 from 100 μm to 30 from 80 μm to 35 or from 60 μm to 40 In some embodiments, the plurality of wells has an average cross-sectional diameter of about 50 μm.

In some embodiments, the hydrogel further comprises a fluorophore. In some embodiments, the hydrogel is luminescent. In certain embodiments, the hydrogel is fluorescent.

In some embodiments, at least some of the plurality of pores are located at the exterior surface of the polymer structure of the hydrogel. In some embodiments, at least some of the plurality of pores are embedded within the polymer structure. In certain embodiments, the plurality of pores are present throughout the polymer structure of the hydrogel.

In some embodiments, at least a portion of the hydrogel can be degraded. In certain embodiments, the hydrogel can be hydrolytically degraded. In some embodiments, the hydrogel can be enzymatically degraded. In some embodiments, the hydrogel can be enzymatically degraded with a plasmin, a matrix metalloproteinase (MMP), a lysozyme, a hyaluronidase, a chondroitinase, heparinase, heparanase, any derivative thereof, or any combination thereof. In some embodiments, the hydrogel can be enzymatically degraded with any enzyme appropriate for said degradation.

This disclosure provides for solutions that can be used in the production of the hydrogels disclosed herein. In some embodiments, the solution for producing the hydrogel comprises from 3 wt % to 6 wt % monomer units, wherein 18% to 35% of the monomer units are cross-linking units. In some embodiments, the solution further comprises a radical initiator (also referred to herein as an initiator). In some embodiments, the solution further comprises a solvent. In certain embodiments, the solution comprises from 3 wt % to 6 wt % monomer units wherein 18% to 35% of the monomer units are cross-linking units, a radical initiator, and a solvent. In some embodiments, the solution further comprises a surfactant. In some embodiments, the solvent comprises water.

In some cases, the solution for producing the hydrogel comprises from 0.1 wt % to 90 wt % monomer units. Among these monomer units, from about 0.1% to about 90% of the monomer units can be cross-linking units. The solution can comprise a radical initiator. The solution can comprise a solvent. The solvent may or may not be organic solvent. In some embodiments, the concentration of the monomer units or %T of the hydrogel is from 1.0 wt % to 3.0 wt %, from 2.0 wt % to 4.0 wt %, from 2.0 wt % to 5.0 wt %, from 3.0 wt % to 4.0 wt %, or from 3.0 wt % to 5.0 wt % in a non-swelled form. In some embodiments, the %T of the hydrogel is at least about 1.0 wt %, 2.0 wt %, 3.0 wt %, 4.0 wt %, 5.0 wt % or more in a non-swelled form. In some embodiments, the concentration of the crosslinking units in the hydrogel is from 10 wt % to 20 wt %, from 10 wt % to 30 wt %, from 10 wt % to 40 wt %, from 10 wt % to 50 wt %, from 10 wt % to 60 wt %, from 20 wt % to 30 wt %, from 20 wt % to 40 wt %, from 20 wt % to 50 wt %, or from 20 wt % to 60 wt % of the total monomer units. In some embodiments, the concentration of the crosslinking units in the hydrogel is at least about 5 wt %, 10 wt %, 20 wt %, 30 wt % or more of the total monomer units. In some embodiments, the concentration of the crosslinking units in the hydrogel is at most about 60 wt %, 50 wt %, 40 wt %, 30 wt %, 20 wt % or less of the total monomer units. In some embodiments, the concentration of the monomer units or %T of the hydrogel is from 1.0% w/v to 3.0% w/v, from 2.0% w/v to 4.0% w/v, from 2.0% w/v to 5.0% w/v, from 3.0% w/v to 4.0% w/v, or from 3.0% w/v to 5.0% w/v in a non-swelled form. In some embodiments, the %T of the hydrogel is at least about 1.0% w/v, 2.0% w/v, 3.0% w/v, 4.0% w/v, 5.0% w/v or more in a non-swelled form. In some embodiments, the concentration of the crosslinking units in the hydrogel is from 10% w/v to 20% w/v, from 10% w/v to 30% w/v, from 10% w/v to 40% w/v, from 10% w/v to 50% w/v, from 10% w/v to 60% w/v, from 20% w/v to 30% w/v, from 20% w/v to 40% w/v, from 20% w/v to 50% w/v, or from 20% w/v to 60% w/v of the total monomer units. In some embodiments, the concentration of the crosslinking units in the hydrogel is at least about 5% w/v, 10% w/v, 20% w/v, 30% w/v or more of the total monomer units. In some embodiments, the concentration of the crosslinking units in the hydrogel is at most about 60% w/v, 50% w/v, 40% w/v, 30% w/v, 20% w/v or less of the total monomer units.

### Methods of Making Hydrogels

This disclosure provides for methods of making the hydrogels disclosed herein. In some embodiments, the method comprises obtaining a solution, wherein the solution comprises from 3 wt % to 6 wt % monomer units, wherein from 18% to 35% of the monomer units are cross-linking units, the solution further comprises a radical initiator and a solvent, and wherein the method comprises using the radical initiator to polymerize the monomer units, and the method further comprises curing the polymer structure. In some embodiments, the method further comprises the step of punching out the polymer structure to form the hydrogel. In some embodiments, the solution further comprises a surfactant. In certain embodiments, the solvent comprises water.

In some embodiments, this disclosure provides for methods of making the hydrogels disclosed herein, wherein the method comprises (i) obtaining a solution, the solution comprising a plurality of monomers, wherein at least one of the monomers is a crosslinking unit, and the solution further comprising a solvent and a radical initiator; (ii) using the radical initiator to polymerize the monomer units; and (iii) curing the polymer structure.

In some embodiments, this disclosure provides for methods of making the hydrogels disclosed herein. The method can comprise obtaining a solution, the solution comprising a plurality of monomers. At least one of the monomers can be a crosslinking unit. The solution can further comprise a solvent and a radical initiator. The solution of monomer units and the solvent can be encapsulated or emulsified in a compartment. The compartment can be a droplet, for example, water-in-oil droplet. In some cases, the oil phase comprise the initiator.

In some embodiments, the solution comprises less than 10 wt %, less than 9 wt %, less than 8 wt %, less than 7 wt %, less than 6 wt %, less than 5 wt %, less than 4 wt %, less than 3 wt %, less than 2 wt %, or less than 1 wt % monomer units. In some embodiments, wt % of the monomer units in the solution is no more than 90 wt %, no more than 80 wt %, no more than 70 wt %, no more than 60 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 9 wt %, no more than 8 wt %, no more than 7 wt %, no more than 6 wt %, no more than wt %, no more than 4 wt %, no more than 3 wt %, no more than 2 wt %, or no more than 1 wt %. In some embodiments, wt % of the monomer units in the solution is from 0.5 wt % to 20 wt %, from 1 wt % to 15 wt %, from 1.5 wt % to 15 wt %, from 1.75 wt % to 12.5 wt %, from 2 wt % to 10 wt %, from 2.5 wt % to 8 wt %, from 3 wt % to 6 wt %, from 0.1 wt % to 90 wt %, from 0.1 wt % to 80 wt %, from 0.1 wt % to 70 wt %, from 0.1 wt % to 60 wt %, from 0.1 wt % to 50 wt %, from 0.1 wt % to 40 wt %, from 0.1 wt % to 30 wt %, from 0.1 wt % to 20 wt %, from 0.1 wt % to 10 wt %, from 0.1 wt % to 1 wt %, from 1 wt % to 90 wt %, from 1 wt % to 80 wt %, from 1 wt % to 70 wt %, from 1 wt % to 60 wt %, from 1 wt % to 50 wt %, from 1 wt % to 40 wt %, from 1 wt % to 30 wt %, from 1 wt % to 20 wt %, from 1 wt % to 10 wt %, or from 1 wt % to 5 wt %. In certain embodiments, the wt % of the monomer units in the solution is from 2.75 wt % to 6 wt %. In certain embodiments, the wt % of the monomer units in the solution is from 3 wt % to 6 wt %. Alternatively, various ranges of the monomer units described herein can be shown in “% w/v” as described herein. For example, in some embodiments, the solution comprises less than 10% w/v, less than 9% w/v, less than 8% w/v, less than 7% w/v, less than 6% w/v, less than 5% w/v, less than 4% w/v, less than 3% w/v, less than 2% w/v, or less than 1% w/v monomer units. In some embodiments, % w/v of the monomer units in the solution is no more than 90% w/v, no more than 80% w/v, no more than 70% w/v, no more than 60% w/v, no more than 50% w/v, no more than 45% w/v, no more than 40% w/v, no more than 35% w/v, no more than 30% w/v, no more than 25% w/v, no more than 20% w/v, no more than 15% w/v, no more than 10% w/v, no more than 9% w/v, no more than 8% w/v, no more than 7% w/v, no more than 6% w/v, no more than % w/v, no more than 4% w/v, no more than 3% w/v, no more than 2% w/v, or no more than 1% w/v. In some embodiments, % w/v of the monomer units in the solution is from 0.5% w/v to 20% w/v, from 1% w/v to 15% w/v, from 1.5% w/v to 15% w/v, from 1.75% w/v to 12.5% w/v, from 2% w/v to 10% w/v, from 2.5% w/v to 8% w/v, from 3% w/v to 6% w/v, from 0.1% w/v to 90% w/v, from 0.1% w/v to 80% w/v, from 0.1% w/v to 70% w/v, from 0.1% w/v to 60% w/v, from 0.1% w/v to 50% w/v, from 0.1% w/v to 40% w/v, from 0.1% w/v to 30% w/v, from 0.1% w/v to 20% w/v, from 0.1% w/v to 10% w/v, from 0.1% w/v to 1% w/v, from 1% w/v to 90% w/v, from 1% w/v to 80% w/v, from 1% w/v to 70% w/v, from 1% w/v to 60% w/v, from 1% w/v to 50% w/v, from 1% w/v to 40% w/v, from 1% w/v to 30% w/v, from 1% w/v to 20% w/v, from 1% w/v to 10% w/v, or from 1% w/v to 5% w/v. In certain embodiments, the % w/v of the monomer units in the solution is from 2.75% w/v to 6% w/v. In certain embodiments, the % w/v of the monomer units in the solution is from 3% w/v to 6% w/v.

As disclosed above, the plurality of monomers in the solution comprise at least one crosslinking unit. As further disclosed above, the amount of crosslinking unit can be represented by %C. In some embodiments, the %C of monomers in the solution is no less than 0.1%, no less than 1%, no less than 5%, no less than 10%, no less than 15%, no less than 16%, no less than 17%, no less than 18%, no less than 19%, no less than 20%, no less than 21%, no less than 22%, no less than 23%, no less than 24%, no less than 25%, no less than 26%, no less than 27%, no less than 28%, no less than 29%, no less than 30%, no less than 31%, no less than 32%, no less than 33%, no less than 34%, no less than 35%, no less than 36%, no less than 37%, no less than 38%, no less than 39%, no less than 40%, no less than 50%, no less than 60%, no less than 70%, no less than 80%, or no less than 90%. In some embodiments, the %C of monomers in the solution is from 0.1% to 90%, from 0.1% to 80%, from 0.1% to 70%, from 0.1% to 60%, from 0.1% to 50%, from 0.1% to 40%, from 0.1% to 30%, from 0.1% to 20%, from 0.1% to 10%, from 0.1% to 5%, or from 0.1% to 1%. In some embodiments, the %C of monomers in the solution is from 1% to 90%, from 1% to 80%, from 1% to 70%, from 1% to 60%, from 1% to 50%, from 1% to 40%, from 1% to 30%, from 1% to 20%, from 1% to 10%, or from 1% to 5%. In some embodiments, the %C of monomers in the solution is from 15% to 40%, from 16% to 39%, from 17% to 38%, from 18% to 37%, from 18% to 36%, from 18% to 35%, from 19% to 34%, from 19% to 33%, from 19% to 32%, from 19% to 31%, from 19% to 30%, from 20% to 29%, from 20% to 28%, from 20% to 27%, from 20% to 26%, or from 20% to 25%. In some embodiments, the %C of monomers in the solution is from 15% to 20%, from 20% to 25%, from 25% to 30%, or from 30% to 35%. In some embodiment, the %C of monomers in the solution is from 15% to 16%, from 16% to 17%, from 17% to 18%, from 18% to 19%, from 19% to 20%, from 20% to 21%, from 21% to 22%, from 22% to 23%, from 23% to 24%, from 24% to 25%, from 25% to 26%, from 26% to 27%, from 27% to 28%, from 28% to 29%, from 29% to 30%, from 30% to 31%, from 31% to 32%, from 32% to 33%, from 33% to 34%, or from 34% to 35%. In certain embodiments, the %C of monomers in the solution is from 18% to 35%. In certain embodiments, the %C of monomers in the solution is from 18% to 25%.

The total monomer concentration and cross-linking component concentration can be adjusted to produce micrometer sized pores. The pore size can decrease as crosslinking unit increases at a low concentration regime. As cross-linking unit concentration reaches a certain value, the pore size of hydrogel may start to increase with crosslinking unit concentrations until hydrogel collapses. Various ranges of monomers or crosslinking monomers can be used as described herein. For example, in some cases, the monomer concentration (e.g., including both crosslinking units and non-crosslinking units) can be from 1.0 wt % to 3.0 wt %, from 2.0 wt % to 4.0 wt %, from 2.0 wt % to 5.0 wt %, from 3.0 wt % to 4.0 wt %, or from 3.0 wt % to 5.0 wt % in a non-swelled form. In some cases, the monomer concentration can be from 3.0 wt % to 4.0 wt %. For example, the monomer concentration can be about 3.0 wt %, 3.25 wt %, 3.5 wt %, 3.6 wt %, 3.65 wt %, 3.7 wt %, 3.75 wt %, 3.8 wt %, 3.85 wt %, or 4.0 wt %. The crosslinking unit (e.g., bis-acrylamide) can range from 0.5 wt % to 1.5 wt %, from 0.6 wt % to 2.0 wt %, from 0.75 wt % to 1.25 wt %, or from 0.8 wt % to 1.5 wt %, and the corresponding non-crosslinking units (e.g., acrylamide) can be from 3.25 wt % to 2.25 wt %, 3.15 wt % to 1.75 wt %, from 3.0 wt % to 2.5 wt %, or from 2.95 wt % to 2.25 wt %. In some cases, the monomer concentration can be from 1.0% w/v to 3.0% w/v, from 2.0% w/v to 4.0% w/v, from 2.0% w/v to 5.0% w/v, from 3.0% w/v to 4.0% w/v, or from 3.0% w/v to 5.0% w/v in a non-swelled form. In some cases, the monomer concentration can be from 3.0% w/v to 4.0% w/v. For example, the monomer concentration can be about 3.0% w/v, 3.25% w/v, 3.5% w/v, 3.6% w/v, 3.65% w/v, 3.7% w/v, 3.75% w/v, 3.8% w/v, 3.85% w/v, or 4.0% w/v. The crosslinking unit (e.g., bis-acrylamide) can range from 0.5% w/v to 1.5% w/v, from 0.6% w/v to 2.0% w/v, from 0.75% w/v to 1.25% w/v, or from 0.8% w/v to 1.5% w/v, and the corresponding non-crosslinking units (e.g., acrylamide) can be from 3.25% w/v to 2.25% w/v, 3.15% w/v to 1.75% w/v, from 3.0% w/v to 2.5% w/v, or from 2.95% w/v to 2.25% w/v.

In some embodiments, the solution further comprises a radical initiator. In certain embodiments, the radical initiator comprises 2-hydroxy-2-methyl-1-phenyl-propan-1-one (Darocur 1173); 2,2-dimethoxy-2-phenylacetophenone (DMPA); 2-hydroxy-2-methylpropiophenone (HOMPP); ammonium persulfate (APS); tetramethylethylenediamene (TEMED); 1-[4-(2-hydroxyethoxy)-phenyl]-2-hydroxy-2-methyl-1-propane-1-one (Irgacure 2959); an organic salt; barium acetate; Borax; camphorquinone; an enzyme; tissue transglutaminase (optionally with calcium chloride); Eosin-Y; any derivative thereof; or any combination thereof.

In some embodiments, the solvent comprises water. In some embodiments, the solvent comprises an alcohol. In some embodiments, the alcohol comprises methanol, ethanol, n-propanol, isopropanol, butanol, 2-butanol, t-butanol, or isobutanol. The solvent may or may not comprise an organic solvent.

In some embodiments, the solution comprises an accelerator. The accelerator can accelerate the rate of formation of free radicals. An accelerator may be an agent which may initiate the polymerization process (e.g., in some cases, via activation of a polymerization initiator) and thus may reduce the time for the hydrogel formation. In some cases, a single accelerator or a plurality of accelerators may be used for polymerization. Careful tuning of acceleration can be important in achieving suitable polymerization reactions. For example, if acceleration is too fast, weight and excessive chain transfer events may cause poor gel structure and low loading of any desired species. If acceleration is too slow, high molecular weight polymers can generate trapped activation sites (e.g., free radicals) due to polymer entanglement and high viscosities. High viscosities can impede diffusion of species intended for loading, resulting in low to no loading of the species. Tuning of accelerator action can be achieved, for example, by selecting an appropriate accelerator, an appropriate combination of accelerators, or by selecting the appropriate accelerator(s) and any stimulus (e.g., heat, electromagnetic radiation (e.g., light, UV light), another chemical species, etc.) capable of modulating accelerator action. Tuning of initiator action may also be achieved in analogous fashion.

An accelerator may be water-soluble, oil-soluble, or may be both water-soluble and oil-soluble. For example, an accelerator may be tetramethylethylenediamine (TMEDA or TEMED), dimethylethylenediamine, N,N, N,′N′-tetramethylmethanediamine, N,N′-dimorpholinomethane, or N,N,N′,N′-Tetrakis(2-Hydroxypropyl)ethylenediamine. For example, an initiator may be ammonium persulfate (APS) or calcium ions. In some cases, an initiator can function as an accelerator. In some cases, the initiator and the accelerator may be the same chemical.

In some embodiments, the solution further comprises a surfactant. In some embodiments, the surfactant comprises an anionic surfactant, a cationic surfactant, a zwitterionic surfactant, a nonionic surfactant, an ampholytic surfactant, any derivative thereof, or any combination thereof. In some embodiments, the surfactant comprises an alkyl sulfate, an alkyl sulfonate, a bile salt, an anionic detergent, 1-oleoyl-rac-glycerol, 2-cyclohexylethyl-beta-d-maltoside, 4-nonylphenyl-polyethylene glycol, 5-cyclohexylpentyl-beta-d-maltoxide, a glycoside, an amprolium hydrochloride, a benzalkonium chloride, a dodecylalkylammonium bromide, a dodecyltrimethylammonium chloride, Girard's reagent, a hexadecylpyridium chloride, a hexadecyltrimethylammonium bromide, Luviquat, an alkylbenzenethonium chloride, a myristyltrimethylammonium bromide, a tetraalkylammonium bromide, any derivative thereof, or any combination thereof.

In some embodiments, using the radical initiator to polymerize the monomer units comprises activating the radical initiator. In some embodiments, the radical initiator is activated by light, and using the radical initiator comprises exposing the solution to a source of light. In some embodiments, the source of light comprises ultraviolet (UV) light. In some embodiments, the source of light is a light emitting diode. In some embodiments, the source of light comprises a wavelength of 365 nm. In some embodiments, the source of light comprises a wavelength from 300 nm to 400 nm, from 400 nm to 700 nm, or from 700 nm to 1,000 nm.

In some embodiments, using the radical initiator comprises using ionizing radiation. In some embodiments, the ionizing radiation comprises a-rays, B-rays, y-rays, x-rays, or any combination thereof.

In some embodiments, using the radical initiator comprises application of friction. In some embodiments, using the radical initiator comprises stirring, sonication, or any combination thereof.

In some embodiments, using the radical initiator to polymerize the monomer units comprises the addition of a compound. In some embodiments, the compound comprises a catalyst. In some embodiments, the compound activates and/or assists the polymerization. In some embodiments, using the radical initiator comprises a chemical reaction. In some embodiments, the chemical reaction comprises a redox reaction. As a non-limiting example, the redox reaction can comprise reduction of hydrogen peroxide by iron. In some embodiments, the chemical reaction comprises the dissociation of a persulfate. In some embodiments, the chemical reaction comprises electrolysis.

In some embodiments, using the radical initiator comprises applying a temperature to the solution. As a non-limiting example, some radical initiators activate upon the application of heat to reach a threshold temperature, whereupon polymerization can take place. In some embodiments, the application of heat comprises warming the solution in order to activate the radical initiator. In some embodiments, the solution is warmed to over 40° C., over 50° C., over 60° C., over 70° C., over 80° C., over 90° C., over 100° C., over 120° C., over 130° C., over 140° C., over 150° C., over 160° C., over 170° C., over 180° C., over 190° C., over 200° C., over 250° C., over 300° C., over 350° C., over 400° C., over 450° C., or over 500° C.

Curing the polymer structure can allow the propagation of cross-linking. In some embodiments, the curing comprises applying a temperature to the polymer structure. In some embodiments, the polymer structure is warmed to over 10° C., over 20° C., over 30° C., over 40° C., over 50° C., over 60° C., over 70° C., over 80° C., over 90° C., over 100° C., over 120° C., over 130° C., over 140° C., over 150° C., over 160° C., over 170° C., over 180° C., over 190° C., over 200° C., over 250° C., over 300° C., over 350° C., over 400° C., over 450° C., or over 500° C. In some embodiments, the polymer structure is warmed to between 10° C. and 50° C., between 50° C. and 100° C., between 100° C. and 150° C., between 150° C. and 200° C., or between 200° C. and 250° C. In some embodiments, the polymer structure is cured at ambient temperature (i.e., between

In some embodiments, curing the polymer structure comprises the application of a source of light. In some embodiments, the source of light comprises ultraviolet (UV) light. In some embodiments, the source of light is a light emitting diode. In some embodiments, the source of light comprises a wavelength of 365 nm. In some embodiments, the source of light comprises a wavelength from 300 nm to 400 nm, from 400 nm to 700 nm, or from 700 nm to 1,000 nm.

In some embodiments, curing the polymer structure comprises the use of a chemical additive. In some embodiments, the chemical additive comprises a sulfur curing system, a peroxide, a metallic oxide, an acetoxysilane, a urethane crosslinker, any derivative thereof, or any combination thereof.

In some embodiments, the method of making the hydrogel further comprises the step of punching out the hydrogel. In some embodiments, the hydrogels are fabricated in a shaped template. In some embodiments, the hydrogels are fabricated in a mold. In some embodiments, hole punch is used to punch out hydrogel samples.

In some cases, the method of making the hydrogel may further comprise deoxygenation. The deoxygenation may ensure reproducibility of each batch of hydrogels. The deoxygenation can be achieved by bubbling Argon gas for at least about 10 min, 15 min, 20 min, 25 min, 30 min, 35 min, 40 min or more in aqueous monomer solution and/or surfactant oil solution before microfluidic generation of droplets.

### Target Molecule

In some embodiments, the hydrogel comprises a target molecule. In some embodiments, the target molecule comprises a protein, a nucleic acid molecule, a peptide, a biomolecule, a drug, a chemical moiety, a lipid, a cell, any derivative thereof, or any combination thereof. The chemical moiety can comprise a glycan. The target molecule can be attached to the hydrogel. In some embodiments, a nucleic acid can be attached to a monomer of the hydrogel. In some embodiments, a nucleic acid can be attached to an acrylamide monomer. In some embodiments, a protein or peptide can be attached to a monomer of the hydrogel. In some embodiments, the protein or peptide can be attached to the monomer by an attachment comprising a disulfide bond. In certain embodiments, a protein or peptide can be attached to acrylamide of the hydrogel by an attachment comprising disulfide bonds.

Nucleic Acid Molecule

In some embodiments, the target molecule comprises a nucleic acid molecule. In some embodiments, the nucleic acid molecule may comprise DNA, RNA, and/or an artificial or synthetic nucleic acid or nucleic acid analog or mimic. For example, in some embodiments, a nucleic acid molecule described herein may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA and/or that targets an expression product), etc. In some embodiments, the nucleic acid molecule encodes a peptide or protein.

In some embodiments, the nucleic acid sequence encodes at least a portion of a polypeptide. The nucleic acid can encode at least a portion of a polypeptide. In certain embodiments, the nucleic acid sequence encodes at least a portion of a polypeptide, and the sequence comprises a cleavable moiety described elsewhere herein.

In some embodiments, a nucleic acid molecule as described herein may include one or more residues that is not a naturally-occurring DNA or RNA residue, may include one or more linkages that is/are not phosphodiester bonds (e.g., that may be, for example, phosphorothioate bonds, etc.), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

In some embodiments, a nucleic acid molecule may comprise one or more nucleoside analogs. In some embodiments, a nucleic acid molecule may include in addition to or as an alternative to one or more natural nucleosides, e.g., purines or pyrimidines, e.g., adenine, cytosine, guanine, thymine and uracil. In some embodiments, a nucleic acid molecule includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-i sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

In some embodiments, the target molecule comprises a nucleic acid molecule that is attached to the hydrogel. In certain embodiments, the nucleic acid molecule is covalently attached to the hydrogel. In some embodiments, the nucleic acid molecule is attached to a monomer of the polymer structure of the hydrogel. In some embodiments, at least one nucleic acid molecule is attached to at least one of the plurality of monomer units of the hydrogel. In some embodiments, a plurality of a nucleic acid molecules is attached to a plurality of monomer units of the hydrogel. In other embodiments, a plurality of a first nucleic acid molecule is attached to a plurality of monomer units of the hydrogel and a plurality of a second nucleic acid molecule is attached to a plurality of monomer units of the hydrogel, wherein the first nucleic acid molecule is different than the second nucleic acid molecule.

In some embodiments, the nucleic acid molecule is a linker that is covalently attached to a monomer. The nucleic acid molecule can be a linker attached to the polymer structure of the hydrogel at one end and another target molecule at the other end, such as a protein.

In some embodiments, the hydrogel comprises (i) a first nucleic acid molecule attached to at least one of the plurality of hydrogel monomer units, the first nucleic acid molecule comprising a first nucleic acid sequence; and (ii) a second nucleic acid molecule attached to at least one of the plurality of hydrogel monomer units, the second nucleic acid molecule comprising a second nucleic acid sequence. In some embodiments, the hydrogel monomer to which the first nucleic acid molecule is attached and the hydrogel monomer to which the second nucleic acid molecule is attached are not the same hydrogel monomer.

In some embodiments, the invention comprises (i) the hydrogel comprising a nucleic acid molecule encoding at least a portion of a polypeptide; and (ii) a protein. The nucleic acid molecule and the protein can be attached. The first nucleic acid molecule and the protein can be attached via protein binding domain to form a target molecule complex. In some embodiments, the protein comprises a multimeric protein. The target molecule complex can be attached to the hydrogel. The target molecule complex can be cleaved from the hydrogel.

Linkers

In some embodiments, the target molecule attaches to the hydrogel through a linker. In some embodiments, a linker comprises a nucleic acid molecule described herein. In some embodiments, the linker comprises an oligonucleotide.

A linker may be a chemical bond, e.g., one or more covalent bonds or non-covalent bonds. In some embodiments, the linker is a covalent bond. In some embodiments, the linker is a non-covalent bond. In some embodiments, a linker is a peptide linker. Such a linker may be between 2-30 amino acids, or longer. In some embodiments, a linker can be used, e.g., to space the hydrogel from the target molecule. In some embodiments, for example, a linker can be positioned between a target molecule and another target molecule. In some embodiments, a linker can be positioned between domains in the target molecule, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release the target molecule from the hydrogel, but sufficiently stable to prevent premature cleavage.

As will be known by one of skill in the art, commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of a linker in aqueous solutions by forming hydrogen bonds with water molecules, and therefore reduce unfavorable interactions between a linker and protein moieties.

Rigid linkers are useful to keep a fixed distance between domains and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP),, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linkers include a hydrophobic linker, such as a negatively charged sulfonate group; lipids, such as a poly (——CH₂——) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a promoting agent (e.g. two polypeptides). Non-covalent linkers are also included, such as hydrophobic lipid globules to which the target molecule is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of target molecule or hydrogel may be linked using charge-based chemistry, such that a positively charged component of the target molecule or hydrogel is linked to a negative charge of another molecule.

In some embodiments, at least one linker is attached to at least one of the plurality of monomer units of the hydrogel. In some embodiments, a plurality of linkers is attached to a plurality of monomer units of a hydrogel.

Cleavage Moiety

In some embodiments, the target molecule comprises a cleavage moiety. In certain embodiments, the cleavage moiety comprises an endoprotease cleavage moiety (i.e., a moiety that can be hydrolyzed by an endoprotease enzyme). In some embodiments, the cleavage moiety comprises a restriction enzyme cleavage site.

For example, the nucleic acid molecule comprises a nucleic acid sequence encoding a protein and a restriction enzyme cleavage site.

In some embodiments, the nucleic acid sequence encodes a cleavable moiety (e.g., a moiety that can be degraded, such as by enzymatic or hydrolytic degradation). In certain embodiments, the nucleic acid sequence encodes an endoprotease cleavage moiety (e.g., a cleavable moiety that is cleaved by an endoprotease enzyme). In some embodiments, the endoprotease cleavage moiety.

In some embodiments, the hydrogel comprises (i) a first nucleic acid molecule comprising a sequence encoding an endoprotease cleavage moiety; and (ii) a second nucleic acid molecule comprising a sequence encoding at least a portion of a polypeptide, wherein the first nucleic acid molecule and the second nucleic acid molecule are attached to the hydrogel. The second nucleic acid can further comprise a restriction enzyme cleavage site. In some embodiments, the second nucleic acid can bind to a protein via a protein binding domain. The protein can be a multimeric protein.

Protein

A target molecule can be a protein, or a nucleic acid molecule attached to a protein. In some embodiments, the target molecule comprises a protein binding domain. In certain embodiments, a linker comprises a protein binding domain. The protein binding domain can be part of a protein binding pair, e.g., avidin/biotin. One of the avidin/biotin pair is part of the target molecule and the other part of the pair can attach to another target molecule.

In some embodiments, the protein is a peptide or protein moiety. In some embodiments, a protein comprises an entire protein. In some embodiments, a protein comprises a protein fragment. In some embodiments, a protein comprises an antibody. In some embodiments, a protein comprises an antibody fragment. As used herein, a protein may comprise an entire protein or a portion or fragment of a protein.

Some examples of a protein include, but are not limited to, fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides. Proteins useful in the invention described herein also include antigen-binding complexes (e.g., MHC, TCR, BCR), antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113).

Some further examples of a protein include a therapeutic protein. In some embodiments, therapeutic proteins that can have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

In some embodiments, a target molecule comprises a protein. The protein can be a multimeric protein, e.g., dimer, trimer, tetramer, etc. In some embodiments, the multimer is a homomultimer, e.g., identical subunits, or homooligomer. In some embodiments, the multimer is a heteromultimer, e.g., different subunits. In certain embodiments, the multimeric protein is a tetrameric protein, also referred to herein as a tetramer.

Cells

In some embodiments, the hydrogel is linked to a target molecule that comprises a cell or a plurality of cells. In some embodiments, the cell(s) is attached to the hydrogel through a linker, e.g., a protein, e.g., a protein multimer, e.g., an antibody or receptor.

Examples of cells may include embryonic stem cells, induced pluripotent stem cells, hematopoietic stem cells, neuronal stem cells, and mesenchymal stem cells, CD4+ T cells, lymphoid progenitor cells, myeloid progenitor cells, macrophages, dendritic cells, gut associated lymphoid tissue cells, hepatocytes, islet cells, CD34+ cells, circulating blood cells, e.g., a reticulocytes, myeloid progenitor cells, bone marrow cells (e.g., a myeloid progenitor cells, erythroid progenitor cells, hematopoietic stem cells, or mesenchymal stem cells), myeloid progenitor cells (e.g. common myeloid progenitor (CMP) cells), erythroid progenitor cells (e.g. megakaryocyte erythroid progenitor (MEP) cells), hematopoietic stem cells (e.g. a long term hematopoietic stem cells (LT-HSC), short term hematopoietic stem cells (ST-HSC), multipotent progenitor (MPP) cells, or lineage restricted progenitor (LRP) cells).

In certain embodiments, the plurality of cells is obtained from a subject. Non-limiting examples of subjects include humans, dogs, cats, mice, rats, pigs and transgenic species thereof. In an exemplary embodiment, the subject is a human. Cells can be obtained from a number of sources, including peripheral blood mononuclear cells, bone marrow, lymph node tissue, spleen tissue, umbilical cord, cancer cells and tumors. In certain embodiments, cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, cells are isolated from peripheral blood. Alternatively, cells can be isolated from umbilical cord. In any event, a specific subpopulation of cells can be further isolated by positive or negative selection techniques.

In certain embodiments, any number of cell lines available in the art, may be used.

Production of Target Molecule

The large porosity of the hydrogels disclosed herein can allow for translation to occur within the hydrogel itself, as opposed to traditional hydrogels which can clog upon translation. Due to the porosity and capability of translating within the hydrogel itself, large scale droplet production can be used. The production of uniform droplets (e.g., by emulsion and/or by vortexing) can provide droplet sizes that are approximately the size of the hydrogel. In some embodiments, the average droplet size is less than 10-times greater, less than 9-times greater, less than 8-times greater, less than 7-times greater, less than 6-times greater, less than 5-times greater, less than 4-times greater, less than 3-times greater, less than 2-times greater, or less than 1.5-times greater than the size of the average hydrogel. In some embodiments, the size of the droplets is very uniform, and said uniformity is dependent on the size of the hydrogel. As a non-limiting example, a droplet that is approximately the size of the hydrogel would require that any translation or IVTT occurring is likely inside the hydrogel. Such translation or IVTT could be toward the production of any molecule. In some embodiments, the average droplet size is from 0.9 to 10, from 0.9 to 9, from 0.9 to 8, from 0.9 to 7, from 0.9 to 6, from 0.9 to 5, from 0.9 to 4, from 0.9 to 3, from 0.9 to 2, from 0.9 to 1.9, from 0.9 to 1.8, from 0.9 to 1.7, from 0.9 to 1.6, from 0.9 to 1.5, from 0.9 to 1.4, from 0.9 to 1.3, from 0.9 to 1.2, or from 0.9 to 1.1 times the size of the average hydrogel.

In some embodiments, the target molecule is produced by polymerase chain reaction (PCR).

In some embodiments, the target molecule is produced by in vitro transcription/translation (IVTT). In certain embodiments, the target molecule is produced by mammalian cells. In some embodiments, the target molecule is a protein produced by mammalian cells. In some embodiments, the target molecule can be produced by a non-mammalian cell. The non-mammalian cell can be a bacterial cell. For example, the bacterial cell can be a Escherichia coli (E. coli) cell. Other bacterial cells that can be used include, but are not limited to, Caulobacter crescentus, Rodhobacter sphaeroides, Pseudomonas putida, Halomonas elongate, Streptomyces lividans, Nocardia lactamdurans, Mycobacterium smegmatis, Corynebacterium glutamicum, Bacillus subtilis, Bacillus brevis, Lactobacillus casei, and Lactobacillus reuteri.

In some embodiments, the hydrogel can attach to a plurality of target molecules. In certain embodiments, the hydrogel can attach to a first target molecule and a second target molecule. In some embodiments, the hydrogel can attach to both the first target molecule and the second target molecule at the same time. In some embodiments, the hydrogel can attach to the first target molecule and the second target molecule at different times. In some embodiments, a first target molecule is produced by PCR and a second target molecule is produced by IVTT.

### Target Molecule Complexes

In some embodiments, a target molecule comprises a complex formed by at least two target molecules. In some embodiments, the target molecule complex is a linker attached to a multimeric protein via a protein binding domain, wherein the linker is attached to a hydrogel monomer, e.g., an acrylamide monomer, and wherein the linker comprises a nucleic acid molecule encoding at least a portion of a polypeptide and cleavage site.

In some embodiments, the target molecule complex can comprise a nucleic acid molecule linked to a protein, e.g., a multimeric protein. The nucleic acid molecule can encode at least a portion of a polypeptide and can be a linker that attaches to a protein, such as through a protein binding domain. The nucleic acid molecule can comprise a cleavage moiety. The cleavage moiety can be a restriction enzyme cleavage site.

In some embodiments, the target molecule complex comprises a target molecule attached to a linker. In some embodiments, the linker comprises a protein binding domain. In certain embodiments, the target molecule complex comprises a nucleic acid attached to the protein binding domain of a linker. In some embodiments, the nucleic acid further encodes at least a portion of a polypeptide. In some embodiments, the target molecule complex comprises a nucleic acid attached to a linker via a protein binding domain.

In some embodiments, the linker comprises a protein binding domain. In certain embodiments, the target molecule complex comprises a nucleic acid attached to the protein binding domain of the linker. In some embodiments, the nucleic acid further encodes at least a portion of a polypeptide. In some embodiments, the target molecule complex comprises a nucleic acid attached to a linker via a protein binding domain.

In some embodiments, the linker comprises a nucleic acid molecule encoding at least a portion of a polypeptide. In certain embodiments, the target molecule complex comprises a nucleic acid attached to the linker, the linker comprising a nucleic acid molecule encoding at least a portion of a polypeptide. In some embodiments, the linker is attached to the target nucleic acid molecule via a protein binding domain.

In some embodiments, the linker comprises a nucleic acid molecule encoding a cleavage moiety. In certain embodiments, the target molecule complex comprises a nucleic acid attached to the linker, the linker comprising a nucleic acid molecule encoding a cleavage moiety. In some embodiments, the linker is attached to the target nucleic acid molecule via a protein binding domain.

In some embodiments, the linker comprises a nucleic acid molecule encoding at least a portion of a polypeptide and a cleavage moiety. In certain embodiments, the target molecule complex comprises a nucleic acid attached to the linker, the linker comprising a nucleic acid molecule encoding at least a portion of a polypeptide and a cleavage moiety.

In certain embodiments, the target molecule complex comprises a protein attached to the protein binding domain of a linker. In some embodiments, the protein attached to the protein binding domain of the linker is a multimeric protein. In some embodiments, the protein attached to the protein binding domain of the linker is a heteromultimeric protein. In some embodiments, the protein attached to the protein binding domain of the linker is a multimeric protein associated with a cell.

In some embodiments, the linker comprises a protein binding domain. In certain embodiments, the target molecule complex comprises a protein attached to the protein binding domain of a linker. In some embodiments, the protein attached to the protein binding domain of the linker is a multimeric protein. In some embodiments, the protein attached to the protein binding domain of the linker is a heteromultimeric protein. In some embodiments, the protein attached to the protein binding domain of the linker is a multimeric protein associated with a cell.

In some embodiments, the linker comprises a nucleic acid molecule encoding at least a portion of a polypeptide. In certain embodiments, the target molecule complex comprises a protein attached to the linker, the linker comprising a nucleic acid molecule encoding at least a portion of a polypeptide. In some embodiments, the protein attached to the linker is a multimeric protein. In some embodiments, the protein attached to the linker is a heteromultimeric protein. In some embodiments, the protein attached to the linker is a multimeric protein associated with a cell. In some embodiments, the linker is attached to the protein via a protein binding domain.

In some embodiments, the linker comprises a nucleic acid molecule encoding a cleavage moiety. In certain embodiments, the target molecule complex comprises a protein attached to the linker, the linker comprising a nucleic acid molecule encoding a cleavage moiety. In some embodiments, the protein attached to the linker comprising a nucleic acid molecule encoding a cleavage site is a multimeric protein. In some embodiments, the protein attached to the linker comprising a nucleic acid molecule encoding a cleavage site is a heteromultimeric protein. In some embodiments, the protein attached to the linker comprising a nucleic acid molecule encoding a cleavage site is a multimeric protein associated with a cell. In some embodiments, the linker is attached to the protein via a protein binding domain.

In some embodiments, the linker comprises a nucleic acid molecule encoding at least a portion of a polypeptide and a cleavage moiety. In certain embodiments, the target molecule complex comprises a protein attached to the linker, the linker comprising a nucleic acid molecule encoding at least a portion of a polypeptide and a cleavage moiety. In some embodiments, the protein attached to the linker is a multimeric protein. In some embodiments, the protein attached to the linker is a heteromultimeric protein. In some embodiments, the protein attached to the linker is a multimeric protein associated with a cell. In some embodiments, the linker is attached to the protein via a protein binding domain.

In some embodiments, the target molecule complex comprises a nucleic acid molecule attached to a protein. In some embodiments, the target molecule complex comprises a nucleic acid molecule attached to a protein via a protein binding domain. In some embodiments, the target molecule complex comprises a nucleic acid molecule attached to a protein via a protein binding domain. In some embodiments, the target molecule complex comprises a nucleic acid molecule encoding at least a portion of a polypeptide and is attached to a protein via a protein binding domain.

In some embodiments, the target molecule complex comprises a nucleic acid molecule attached to a multimeric protein via a protein binding domain. In some embodiments, the target molecule complex comprises a nucleic acid molecule encoding at least a portion of a polypeptide and is attached to a multimeric protein via a protein binding domain.

In some embodiments, the target molecule complex comprises a nucleic acid molecule attached to a heteromultimer via a protein binding domain. In some embodiments, the target molecule complex comprises a nucleic acid molecule comprising encode at least a portion of a polypeptide and is attached to a heteromultimer via a protein binding domain.

In some embodiments, the target molecule complex comprises a nucleic acid molecule attached to a multimeric protein via a protein binding domain, wherein the multimeric protein is associated with a cell. In some embodiments, the target molecule complex comprises a nucleic acid molecule encoding at least a portion of a polypeptide and is attached to a multimeric protein via a protein binding domain, wherein the multimeric protein is associated with a cell.

In some embodiments, the target molecule complex comprises a nucleic acid molecule as disclosed herein. In some embodiments, the target molecule complex comprises a protein as disclosed herein. In some embodiments, the target molecule complex comprises a linker as disclosed herein. In some embodiments, the target molecule complex comprises at least one of a nucleic acid molecule, a nucleic acid molecule encoding at least a portion of a polypeptide, a multimeric protein, a heteromultimeric protein, a multimeric protein attached to a cell, a linker, a linker comprising a nucleic acid molecule encoding at least a portion of a polypeptide, a linker comprising a nucleic acid molecule encoding a cleavage moiety, a linker comprising a nucleic acid molecule encoding at least a portion of a polypeptide and a cleavage moiety, any derivative thereof, or any combination thereof. As provided herein, any of the aforementioned target molecule complex moieties can be attached to each other via a protein binding domain, a disulfide bond, a biotin, any derivative thereof, or any combination thereof.

### Methods of Using Hydrogels

This disclosure further provides methods of using hydrogels to produce target molecule complexes as described herein. A target molecule complex can comprise a nucleic acid molecule attached to a protein. A target molecule complex can comprise a nucleic acid molecule attached to a protein via a protein binding domain. The nucleic acid molecule can comprise restriction enzyme cleavage site. The protein can be a multimeric protein, such as a heteromultimer.

In some embodiments, the methods described herein produce target molecule complexes that are attached to the hydrogel. In some embodiments, the methods described herein produce target molecule complexes that are cleaved from the hydrogel.

In some embodiments, a method of using the hydrogel comprises providing the hydrogel and a nucleic acid molecule. In some embodiments, the single compartment comprises the hydrogel and a plurality of nucleic acid molecules.

In some embodiments, the method comprises the step of attaching a nucleic acid molecule to the hydrogel. In some embodiments, the nucleic acid molecule is attached to the hydrogel through covalent bonding, non-covalent bonding, or any combination thereof. In certain embodiments, the target molecule is covalently attached to the hydrogel. In certain embodiments, the target molecule is a nucleic acid molecule. The nucleic acid molecule can be a linker attached to the hydrogel. In some embodiments, the linker comprises a protein binding domain.

The method can further comprise the step of performing IVTT on the nucleic acid molecule to produce a protein or a peptide. The produced protein can bind to the nucleic acid molecule via a protein binding domain to form the target molecule complex attached to the hydrogel. The nucleic acid molecule can comprise a cleavage moiety, such as a restriction enzyme cleavage site. In some embodiments, the method can further comprise adding a restriction enzyme to cleave at the restriction enzyme cleavage moiety to produce a target molecule complex. In some cases, the produced peptide can bind to a target molecule complex, which may be attached to the hydrogel.

### EXAMPLES

The following examples are included to further describe some aspects of the present disclosure, and should not be used to limit the scope of the invention.

### Example 1

### Production of a Porous Hydrogel

This Example demonstrates the production of a porous hydrogel.

Hydrogels were produced by mixing acrylamide monomer units and bis-acrylamide crosslinker units at a variety of relative concentrations, and incubating the mixture until crosslinking was complete.

In this Example, the pre-crosslinked aqueous mix included 45 μL of 40% 1:19 Bis-Acrylamide:Acrylamide stock solution, 32.5 μL of 40% Acrylamide, 10 μL of 10% Ammonium Persulfate (APS), 50 μL of Tris-buffered saline buffer supplemented with 10 mM EDTA and 0.1% Triton X-100 (TEBST). All reagents of the aqueous mixture were combined and stirred. The mixture was emulsified in fluorinated oil with 1.5% TEMED and 1% of 008-FluoroSurfactant, incubated at room temperature for lhr, and then transferred into an oven at 60° C. for overnight incubation, thus forming the hydrogels.

The hydrogels were washed once with 20% 1H,1H,2H,2H-perfluoro-1-octanol (PFO), then washed three times with TEBST, and then washed three times with low TE (1 mM Tris-Cl pH 7.5, 0.1 mM EDTA).

In this Example, the acrylamide final concentration was varied from about 2.65% to about 5.8% of the total weight percentage; the bis-acrylamide final concentration was varied from about 0.2% to about 0.85% of the total weight percentage. Sample 1 comprised approximately 5.8% acrylamide and 0.2% bis-acrylamide by total weight percentages; Sample 2 comprised approximately 3% acrylamide and 0.75% bis-acrylamide by total weight percentages; Sample 3 comprised approximately 2.65% acrylamide and 0.85% bis-acrylamide by total weight percentages. In addition, the crosslinker initiator final concentration was varied from about 0.2% to about 2% APS.

Hydrogels were stored in TEBST at 4° C. until use.

### Example 2

### Characterization of the Hydrogel

This Example demonstrates structural characterization of porous hydrogel.

To determine diffusion capacity of the hydrogels produced in Example 1 displayed, the hydrogels were measured for diffusion of fluorescein isothiocyanate (FITC)-labeled 500 kDa dextran buffer. The hydrogels were incubated at 60° C. on a rotator overnight in 0.1 μM FITC-dextran in TEBST buffer (1× TBS, 10 mM EDTA, 0.1% triton X-100). After incubation, the hydrogels were imaged for diffusion of FITC labeled dextran. Impermeability was measured as a ratio of intensities, R, wherein:

$$R = \frac{{I}_{B}}{{I}_{HG}} \qquad ( eq . 3 )$$

and wherein R is inversely related to the rate of diffusion inside the hydrogel. R is equal to the ratio between the fluorophore background intensity (IB) and the intensity of the fluorophore inside the hydrogel (IHG). A low ratio (e.g., as R approaches 1) corresponds with increased diffusion, and a larger average pore size.

The impermeability of three hydrogel samples were measured. Sample 1 had 6% total acrylamide monomer weight, with 3% of said monomer weight being composed of the bis-acrylamide cross-linking monomer. Sample 2 had 3.75% total acrylamide monomer weight, with 20% of said monomer weight being composed of the bis-acrylamide cross-linking monomer. Sample 3 had 3.5% total acrylamide monomer weight, with 24.3% of said monomer weight being composed of the bis-acrylamide cross-linking monomer. Compositions and Impermeability (R) are provided in Table 1, below.

TABLE 1

| | wt % | Percent cross- | Imperm- |
|---|---|---|---|
| Sample | monomer | linking | eability |
| ID | (% T) | monomer (% C) | (R) |
| Sample 1 | 6% | 3% | 14 |
| Sample 2 | 3.75% | 20% | 3.5 |
| Sample 3 | 3.5% | 24.3% | 1.8 |

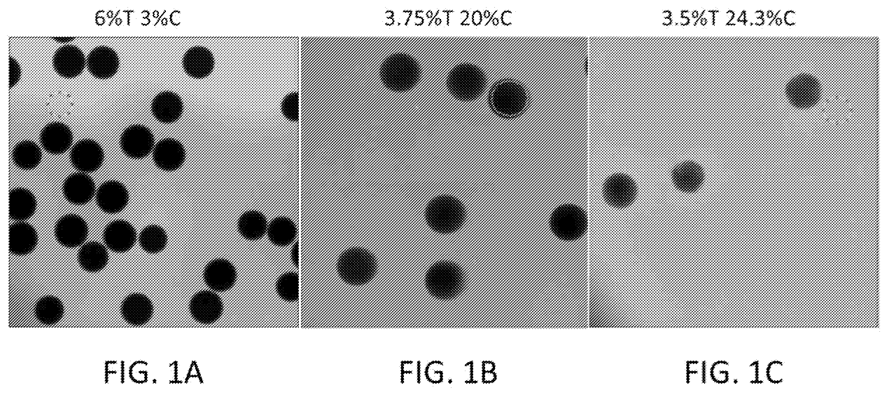

Sample 1, having higher acrylamide total weight (%T =6) and lower weight percentage of bis-acrylamide in total monomer (%C=3) had an impermeability ratio R=14. The high level of impermeability against the background can be seen in FIG. 1A. In comparison, FIG. 1B shows hydrogels from Sample 2, having a decreased total acrylamide weight (%T =3.75) but a significantly increased weight percentage of the crosslinking bis-acrylamide (%C=20), which resulted in Sample 2 having an impermeability ratio of R=3.5. Finally, Sample 3 had an even lower acrylamide total weight (%T=3.5) and higher crosslinking bis-acrylamide weight percentage (%C=24.3), resulting in a decreased impermeability ratio (R=1.8). The low level of impermeability compared to the background can be seen in FIG. 1C.

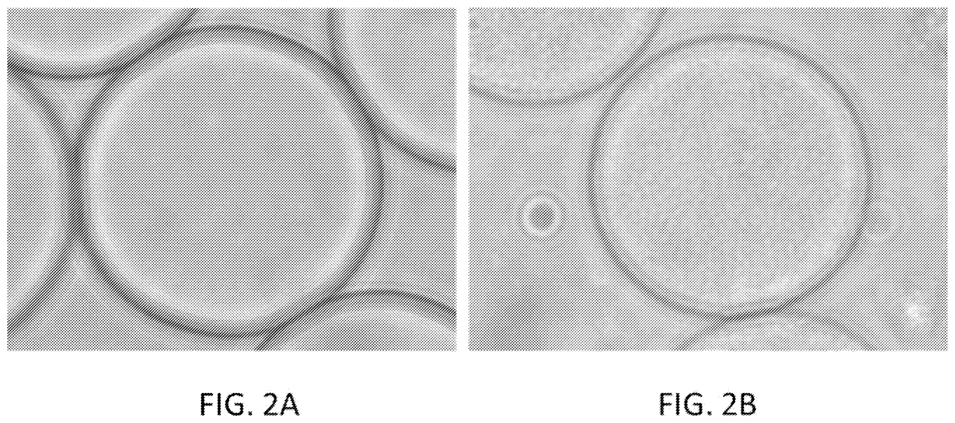

To further determine and measure porosity, the hydrogels were visualized under an optical microscope for visual differences. As shown in FIG. 2A, hydrogels produced from Sample 1 (6% T, 3% C) had a smooth appearance. This is in contrast to hydrogels produced from Sample 2 (3.75% T, 20% C), which were more punctate as seen in FIG. 2B.

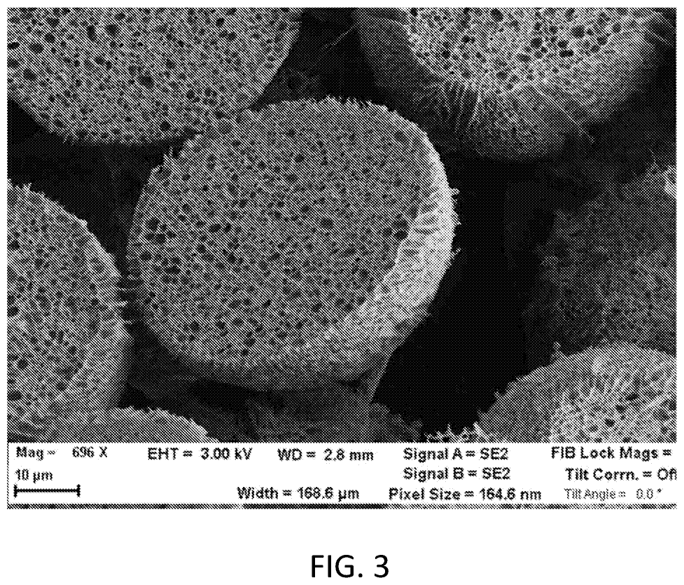

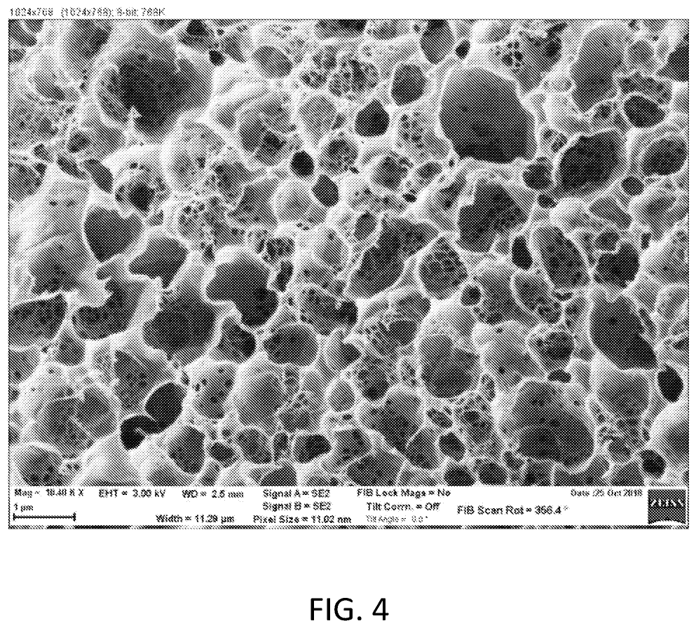

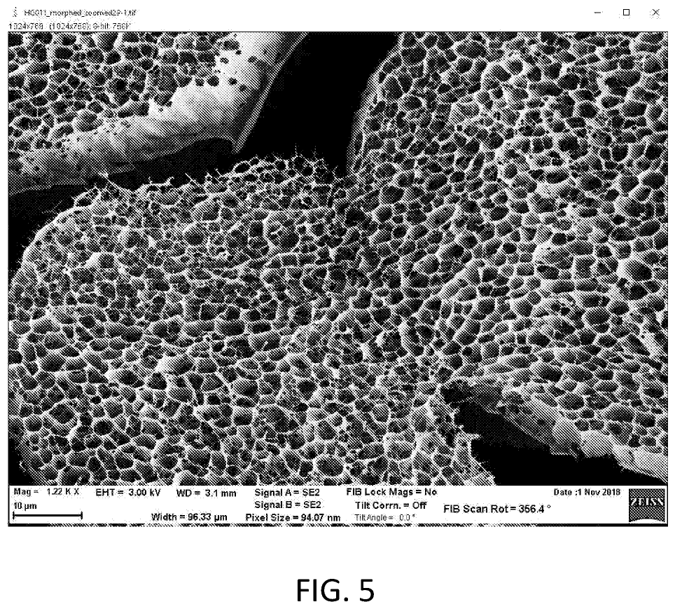

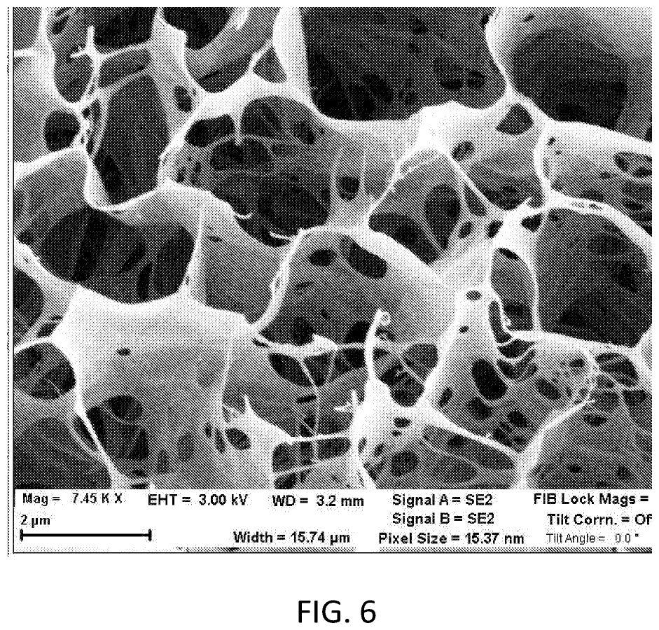

To further measure porosity, Sample 1 and Sample 2 hydrogels were visualized under electron microscopy and pore size was determined. Hydrogels were cryo-frozen at -150° C. to -135° C. and coated with 10 nm of Pt/Pd alloy before imaging with a Scanning Electron Microscope. FIG. 3 shows a 696-fold magnification of hydrogels from Sample 1. Likewise, FIG. 4 shows a 10,400-fold magnification of a hydrogel from Sample 1. These figures reveal that the hydrogels of Sample 1 have an average pore size ranging from about 10 nm to about 100 nm. The hydrogels from Sample 2 were similarly imaged. FIG. 5 shows a 1,220-fold magnification of hydrogels from Sample 2. Likewise, FIG. 6 shows a 7,450-fold magnification of a hydrogel from Sample 2. These figures reveal that the hydrogels of Sample 2 have an average pore size ranging from about 50 nm to about 1 μm. Accordingly, this example shows that increasing the percentage of cross-linking monomers can increase hydrogel pore size, and therefore can increase the diffusion of molecules into and out of the hydrogels.

### Example 3

### Moiety Attachment to Hydrogel

This Example describes the selective attachment of a moiety to the porous hydrogel. This can be a useful feature for delivery or other modes of administration of the moiety.

In this particular Example, nucleic acids were attached to a hydrogel. However, other moieties may be envisioned as attached to the hydrogel (e.g., peptides, chemicals, or any combination thereof). The acrylamide monomer was modified with a solid phase attachment of an oligonucleotide. The oligonucleotide had a 5′ terminal acrylamide modification. These acrylamide monomers were commercially obtained or produced as in Rehman et al., Nucleic Acids Res, 27(2):649-655. The modified monomers were used to produce porous hydrogels as described in Example 1.

In another embodiment of this Example, proteins were attached to a hydrogel. The acrylamide monomer was modified with a linker (e.g., a nucleic acid), which was attached to a protein binding domain (e.g., biotin). These monomers were used to produce the porous hydrogels as described in Example 1. Multimeric proteins produced by in vitro transcription translation (IVTT) or produced in mammalian cells (or E. coli cells) were bound to the hydrogel through the protein binding domain via incubation for one hour at room temperature. Unbound multimeric proteins were then removed by a series of three washes in PBS +0.05% Tween-20. In another embodiment, the hydrogel was modified by the addition of disulfide bonds to attach a multimeric protein.

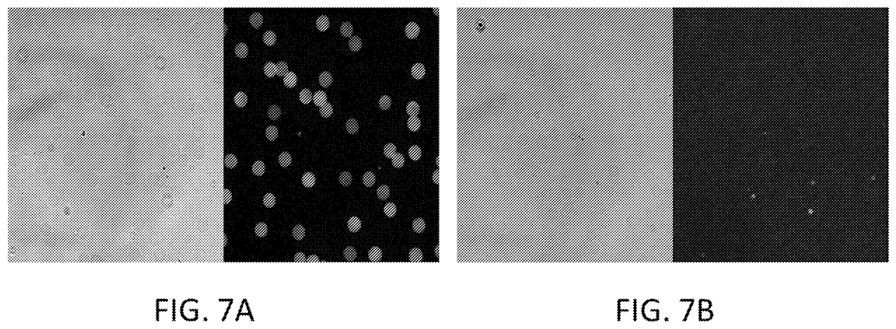

To assess multimeric protein attachment, the hydrogels were stained with Alexa Fluor-488 conjugated anti-multimeric antibodies for one hour at room temperature in darkness. Control hydrogels (e.g., hydrogels having the same %T and %C values, but without the linker and protein binding domain modification) were also combined with Alexa Fluor-488 conjugated anti-multimeric antibodies for one hour at room temperature in darkness. From both samples, free antibody was removed by a series of three washes in PBS +0.05% Tween-20. The resulting hydrogels were imaged using confocal microscopy. FIG. 7A shows the modified hydrogels (e.g., having a nucleic acid linker and biotin). The left panel of FIG. 7A shows the bright field imagery of the hydrogels, while the right panel of FIG. 7A shows the fluorescence imagery captured by the confocal microscopy. The observed fluorescence confirms the antibodies attached to the modified hydrogels. In contrast, FIG. 7B shows the control hydrogels, wherein the left panel shows the bright field imagery and the right panel shows the fluorescence imagery. The observed lack of fluorescence with the control hydrogels confirms that the antibodies would not attach adequately to a hydrogel without a linker and protein binding domain.

### Example 4

### Moiety Cleavage from Hydrogel

This Example demonstrates cleavage of a target moiety from a porous hydrogel.

This Example describes the selective removal of a target moiety from the hydrogel. This can be a useful feature for delivery or other modes of therapeutic administration, and may be useful for generation of target molecule complexes (e.g., soluble target molecule complexes) with specific nucleic acid identifiers. In this Example, the oligonucleotide moiety described in Example 3 which was attached to the acrylamide monomer included a restriction enzyme site. After production of the porous hydrogel, the oligonucleotide was cleaved from the hydrogel with a restriction enzyme (treatment with a restriction enzyme followed the manufacturer's instructions). In some particular embodiments, the restriction enzyme site and placement of the site can be engineered depending on the desired cleavage products (e.g., overhangs left on the hydrogel or overhangs left on the cleaved oligonucleotide).

In an embodiment of this Example, the multimeric protein described in Example 3 was cleaved from the hydrogel. A restriction enzyme site was engineered between the acrylamide monomer and the protein binding domain of the linker (i.e., between the acrylamide and the biotin). Treatment with a restriction enzyme according to the manufacturer's instructions resulted in cleavage of the linker to produce the multimeric protein separated from the hydrogel. In another embodiment of this Example, the multimeric protein was attached to the hydrogel through disulfide bonds and was cleaved by exposing the hydrogel to DTT to reduce the disulfide bonds.

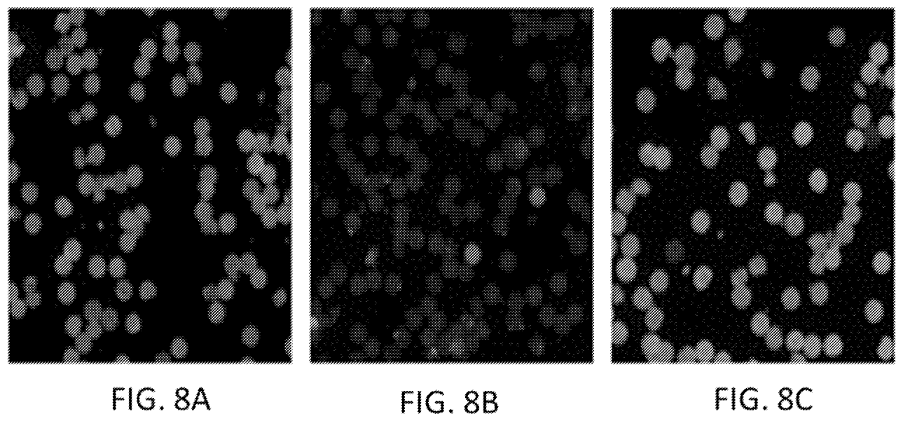

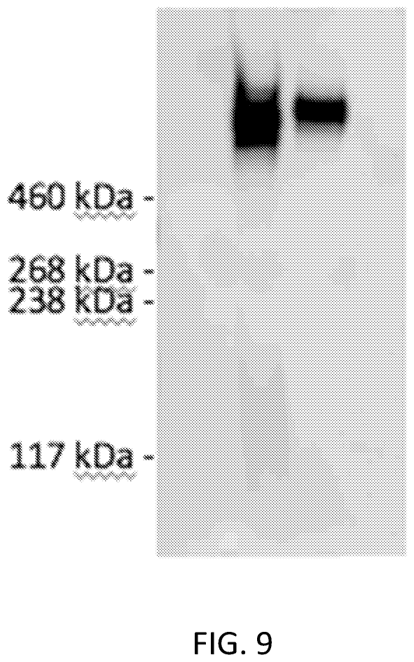

As provided by Example 3, multimeric proteins were attached to the hydrogels. After cleavage treatments as described below, hydrogels were stained with Alexa Fluor-488 conjugated anti-multimeric antibodies for one hour at room temperature in darkness. Free antibody was removed by a series of three washes in PBS +0.05% Tween-20. The hydrogels were imaged using confocal microscopy. FIG. 8A shows the hydrogels having a cleavable linker attached to a fluorescent multimeric protein. FIG. 8B shows a portion of the hydrogels, following treatment with a restriction enzyme, followed by a wash. FIG. 8C shows a portion of the hydrogels that were not treated with a restriction enzyme, followed by a wash. This observation shows the restriction enzyme is capable of cleaving the linker, thus releasing multimer proteins attached to the hydrogel. Following cleavage of the multimeric proteins, the cleaved moieties were analyzed by Western blot. Samples were separated on a 3-8% Tris-Acetate gel and transferred to a nitrocellulose membrane, followed by staining with HRP-conjugated anti-multimeric antibodies, as shown at FIG. 9. This confirmed the cleaved multimeric protein had the desired size.

### Example 5

### Production of a Porous Hydrogel

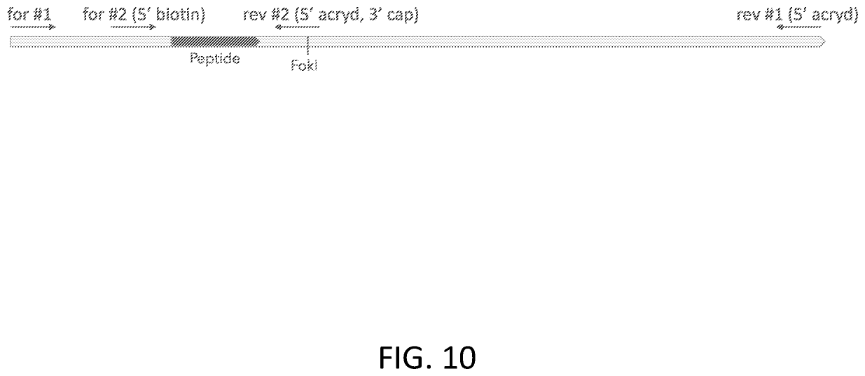

This Example demonstrates production of porous hydrogels that can be used in compositions and methods of the disclosure. Hydrogel beads were produced by mixing acrylamide monomer units and bis-acrylamide crosslinker units at a variety of relative concentrations along with a mixture of acrydated oligonucleotide primers, encapsulating in droplets using a microfluidic drop-maker, and incubating the mixture until crosslinking was complete. In this Example, the pre-crosslinked aqueous mix included 0.75% bis-acrylamide, 3% acrylamide, 5₁1A4 5′-acrydated reverse primer # 1, 25 μM 3′-capped (phosphorylated) and 5′-acrydated reverse primer # 2 (FIG. 10), 0.5% ammonium persulfate, in 10% TEBST (Tris-EDTA-buffered saline plus Tween-20). Primers can be designed to include sequences for enzymatic cleavage, for example sequences targeted by a restriction enzyme, to allow liberation of part of the primer from the hydrogel. Any suitable restriction enzyme can be used. In this example, reverse primer 1 included an Xhol digestion site and reverse primer 2 included a FokI digestion site. All reagents of the aqueous mixture were combined and stirred in a container and the container was put in a chamber with a small opening. Argon gas was used to bubble the aqueous mixture for 30 minutes, then the chamber was sealed completely. At the same time, the oil mixture comprising 1.5% TEMED and 1% of 008-FluoroSurfactant in HFE 7500 oil, was also bubbled with Argon gas for 30 minutes in a chamber. The aqueous mixture was supplemented with the oil mixture, encapsulated in droplets through a microfluidic drop maker. The collected droplets were incubated at room temperature for lhr, and then transferred into an oven at 60° C. for overnight incubation, thus forming the hydrogels. The hydrogel beads were washed once with 20% 1H,1H,2H,2H-perfluoro-1-octanol (PFO), then washed three times with TEBST, and then washed three times with low TE (1 mM Tris-Cl pH 7.5, 0.1 mM EDTA). Hydrogel beads were stored in TEBST at 4° C. until use.

### Example 6

### PCR of Full-Length Antigen-Encoding Templates Onto Hydrogels (PCR1)

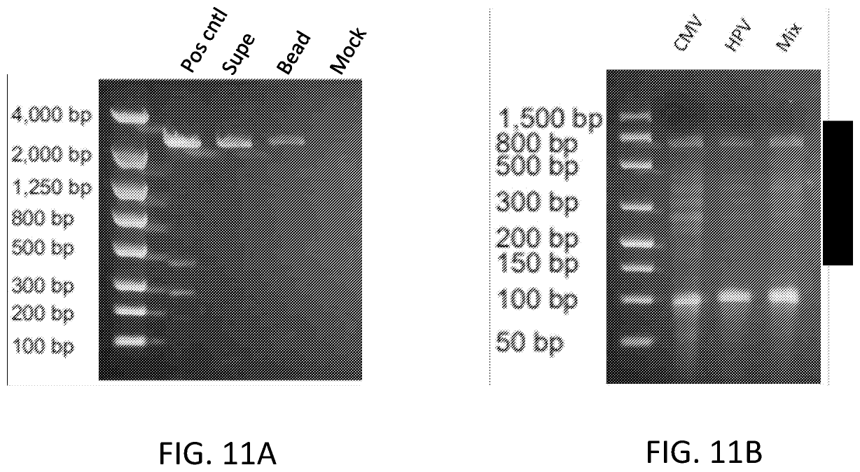

This Example demonstrates PCR of full length antigen-encoding templates onto hydrogels. Linear DNA templates encoding single chain multimeric peptide-MHC were PCR-amplified onto hydrogel beads in drops under single template conditions, where each drop gets at most a single DNA template. 1.4 mL hydrogel beads produced in Example 5 were mixed together with PCR components as follows in a 2mL reaction volume: 400 μL Q5 reaction buffer (New England Biolabs), 40 μL 10 mM dNTP, 40 μL 25 μM forward primer # 1, 40 μL 1μM of non-acrydated reverse primer # 1 (FIG. 10), 40 pL 0.1 pg/ul linear DNA template (or mix of templates), 8 μL 20% IGEPAL, and 20 pL Q5 DNA polymerase (New England Biolabs). The mixture was encapsulated in drops and subjected to 35 cycles of PCR. After drop lysis by addition of an equal volume of 100% perfluorooctanol (PFO), hydrogels were washed with 10 volumes of low TE five times. Aliquots (10 μL ea) of hydrogel beads were digested with a restriction enzyme that cuts within reverse primer # 1 (in this example, Xhol) for 1 h at 37° C., and run on a 1.2% agarose gel to quantify yield and quality of amplicons on hydrogels. As shown in FIG. 11A, full length antigen-encoding templates were PCR amplified onto hydrogels (“bead”).

### Example 7

### PCR of an Identifier (PCR2)

This Example demonstrates PCR amplification of identifiers on hydrogels generated in examples 5 and 6. Any suitable identifier disclosed herein can be used. In this example, a self-identifier was used that corresponds to all or a part of a nucleic acid sequence that encodes the peptide that it identifies. Washed hydrogel beads after PCR1 were digested with shrimp alkaline phosphatase (New England Biolabs) to remove the 3′ cap on reverse primer # 2 and then further washed 5 times with 10 volumes of low TE. 300 μL hydrogel beads were mixed together with PCR components as follows in a 400 μL reaction volume: 80 μL Q5 reaction buffer (New England Biolabs), 8μL 10 mM dNTP, 8 μL 25 μM 5′-biotinylated forward primer # 2, 1.6 μL 20% IGEPAL, and 4 μL Q5 DNA polymerase (New England Biolabs). The mixture was encapsulated in drops and subjected to 20 cycles of PCR. After drop lysis by addition of an equal volume of 100% PFO, hydrogel beads were washed five times with 10 volumes of low TE. Small aliquots of hydrogel beads were digested with a restriction enzyme that cuts within reverse primer 2 (in this example, FokI) for 1 h at 37° C. and run on a 1.2% agarose gel to quantify the yield and quality of identifier amplicons on hydrogels. As shown in FIG. 11B, identifiers were PCR amplified onto hydrogens (“self-identifying nucleic acid”). Three separate bead preps were analyzed: one with a template corresponding to a CMV peptide, one with an HPV peptide, and one with a mixture of templates encoding both peptides (mix). The self-identifying nucleic acid fragment produced by PCR2 is indicated at ˜100 bp.

### Example 8

### In Vitro Transcription/Translation (IVTT) of Single Chain Multimeric Peptide-MHC

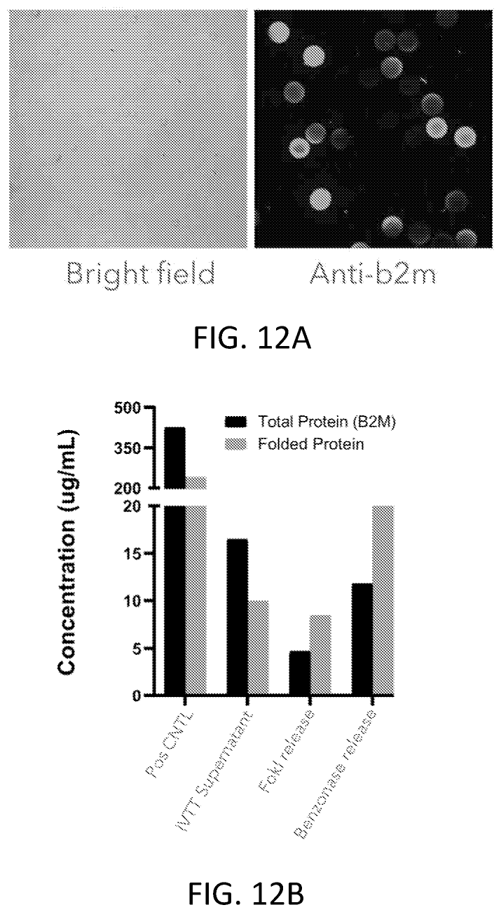

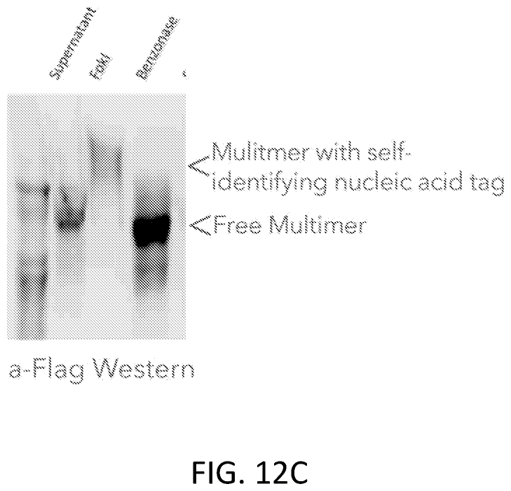

This Example demonstrates single chain peptide-MHC can be in vitro transcribed and translated, for example, using antigen-encoding DNA templates on hydrogels as generated in examples 5 and 6. 120 μL of hydrogel beads were co-encapsulated in drops with 240 μL of IVTT master mix, including 120 μL PURExpress solution A (New England Biolabs), 90 μL PURExpress solution B (NEB), 6 μL RNAse OUT (Invitrogen), 12 μL each Disulfide Bond Enhancer # 1 and # 2 (NEB), and 12 μL Ulpl protease (Invitrogen). Drops were incubated at 22° C. for 20 hours, without shaking. D-Biotin was added to the IVTT reactions to a final concentration of 500 μM prior to breaking drops by addition of an equal volume of 100% PFO. Hydrogel beads were washed five times with 10 volumes of PBS plus 2% BSA. An aliquot of hydrogels was subjected to immunofluorescent staining with 1:10 dilution of Alexa-488-labeled anti-beta-2-microglobulin (B2M) antibody (R&D Systems) in PBS plus 2% BSA for 1 hour at room temperature, followed by five 10-fold washes in PBS plus 2% BSA, and imaging by confocal microscopy (Imagexpress Micro, Molecular Devices, FIG. 12A). Staining was observed in 21% of beads, confirming single template conditions in PCR1, and successful production of single chain peptide-MHC.

### Example 9

### Release and Analysis of Identifier-Tagged Single Chain Multimeric Peptide-MHC from Hydrogels

This Example demonstrates release of folded, identifier-tagged single chain peptide-MHC (sc-pMHC) multimers from hydrogels. sc-pMHC were generated using the methods of examples 5, 6, 7, and 8. The sc-pMHC multimers were bound to the hydrogels via DNA identifiers. sc-pMHC bound to hydrogels via DNA can be released from the hydrogels by digestion with any suitable nuclease. In this example, DNA was digested by benzonase (a non-specific endonuclease) or FokI (a restriction enzyme) in Cutsmart Buffer (NEB), with a 20 hour incubation at 22° C. Protein released by digestion was tested by ELISA to determine yield and folding. Detection was done with a 1:1333 dilution of either HRP-conjugated anti-B2M (Biolegend) or a conformationally sensitive anti-HLA antibody (Santa Cruz), with an HEK-produced sc-pMHC as a standard. ELISA confirmed release of highly folded sc-pMHC multimer (FIG. 12B). Protein released by digestion was also tested by Western Blot, with electrophoresis on a 3-8% Tris-Acetate gel, blotting to nitrocellulose, blocking with PBS plus 3% BSA, and detection with 1μg/mL rat anti-Flag (Biolegend) primary and 1:1000 Alexa647 conjugated Anti-Rat IgG secondary (Invitrogen). The slow migration of FokI-released sc-pMHC multimer relative to benzonase-released sc-pMHC, or relative to supernatant from the in vitro transcription/translation supernatant, demonstrates successful tagging of the sc-pMHC with a nucleic acid identifier (FIG. 12C).

What is claimed is:

1. A hydrogel, the hydrogel comprising:
a polymer structure, wherein:
the polymer structure comprises a plurality of monomer units; and
at least one of the monomer units is a cross-linking unit; and
a plurality of pores in the polymer structure, the plurality of pores having an average pore size from 0.1 μm to 60 μm.

2. The hydrogel of claim 1, wherein the pore size is from 1 μm to 50 μm.

3. The hydrogel of any one of claims 1-2, wherein the plurality of monomer units is from 0.1 wt % to 90 wt % of the hydrogel.

4. The hydrogel of any one of claims 1-3, wherein the plurality of monomer units is from 2.75 wt % to 6 wt % of the hydrogel.

5. The hydrogel of any one of claims 1-4, wherein from 0.1% to 90% of the monomer units are cross-linking units.

6. The hydrogel of any one of claims 1-5, wherein from 18% to 35% of the monomer units are cross-linking units.

7. The hydrogel of any one of claims 1-6, wherein at least one linker is attached to at least one of the plurality of monomer units.

8. The hydrogel of any one of claims 1-7, wherein the polymer structure further comprises a target molecule attached to at least one linker.

9. The hydrogel of any one of claims 7-8, wherein the linker comprises a nucleic acid.

10. The hydrogel of any one of claims 7-9, wherein the linker comprises an oligonucleotide.

11. The hydrogel of any one of claims 7-10, wherein the linker is covalently attached to the at least one monomer unit.

12. The hydrogel of any one of claims 7-11, wherein the linker further comprises a protein binding domain.

13. The hydrogel of any one of claims 8-12, wherein the target molecule comprises a protein, a nucleic acid molecule, a peptide, a biomolecule, a drug, a chemical moiety, a lipid, any derivative thereof, or any combination thereof.

14. The hydrogel of any one of claims 8-13, wherein the target molecule is a nucleic acid molecule, and wherein the target molecule is covalently attached to the linker.

15. The hydrogel of any one of claims 8-14, wherein the target molecule is a multimeric protein.

16. The hydrogel of any one of claims 8-15, wherein the target molecule is a heteromultimer.

17. The hydrogel of any one of claims 15-16, wherein the multimeric protein is produced by in vitro transcription/translation (IVTT).

18. The hydrogel of any one of claims 15-16, wherein the multimeric protein is produced in mammalian cells or bacterial cells.

19. The hydrogel of any one of claims 12-18, wherein the target molecule is attached to the protein binding domain.

20. The hydrogel of any one of claims 1-6, wherein the polymer structure further comprises:
a first nucleic acid molecule attached to at least one of the plurality of monomer units, the first nucleic acid molecule comprising a first sequence; and
a second nucleic acid molecule attached to at least a one of the plurality of monomer units, the second nucleic acid molecule comprising a second sequence.

21. The hydrogel of claim 20, wherein the first sequence encodes a cleavage moiety.

22. The hydrogel of any one of claims 20-21, wherein the first sequence encodes an endoprotease cleavage moiety.

23. The hydrogel of any one of claims 20-21, wherein the second sequence encodes at least a portion of a polypeptide.

24. The hydrogel of any one of claims 1-23, wherein the hydrogel has a diameter less than 250 μm.

25. The hydrogel of any one of claims 1-24, wherein the plurality of monomer units comprise a PEG monomer, an acrylamide monomer, a PEGDA monomer, a chitosan, an alginate, a gelatin, a hyaluronic acid, a chondroitin, a fibrinogen, a peptide, a polyfumerate, a phosphoester, any derivative thereof, or any combination thereof.

26. The hydrogel of any one of claims 1-25, further comprising a fluorophore.

27. The hydrogel of any one of claims 1-26, wherein the plurality of pores is located at the exterior surface of the polymer structure.

28. The hydrogel of any one of claims 1-27, wherein the plurality of pores is embedded within the polymer structure.

29. The hydrogel of any one of claims 1-28, wherein at least a portion of the hydrogel can be enzymatically degraded.

30. The hydrogel of any one of claims 1-29, wherein at least a portion of the hydrogel can be hydrolytically degraded.

31. A solution for producing the hydrogel of any one of claims 1-30, the solution comprising:
from 0.1 wt % to 90 wt % monomer units, wherein from 0.1% to 90% of the monomer units are cross-linking units;
a radical initiator; and
a solvent.

32. The solution of claim 31 comprising from 2.75 wt % to 6 wt % monomer units.

33. The solution of any one of claims 31-32, wherein from 18% to 35% of the monomer units are cross-linking units.

34. The solution of any one of claims 31-33, further comprising a surfactant.

35. The solution of any one of claims 31-34, wherein the solvent comprises water.

36. A method of making the hydrogel of any one of claims 1-30, the method comprising:
obtaining a solution, the solution comprising:
from 0.1 wt % to 90 wt % monomer units, wherein from 0.1% to 90% of the monomer units are cross-linking units;
a radical initiator; and
a solvent;
using the radical initiator to polymerize the monomer units; and
curing the polymer structure.

37. The method of claim 36, wherein the solution comprises from 2.75 wt % to 6 wt % monomer units.

38. The method of any one of claims 36-37, wherein from 18% to 35% of the monomer units are cross-linking units.

39. The method of any one of claims 36-38, further comprising the step of punching out polymer structure.

40. The method of any one of claims 36-39, wherein the solvent comprises water.

41. The method of any one of claims 36-40, wherein the solution further comprises a surfactant.

42. The method of any one of claims 36-41, wherein the solution is within a droplet.

43. The method of any one of claims 36-42, further comprising generating a droplet comprising the solution.

44. The method of claim 43, further comprising curing the polymer structure within the droplet.

45. A method of producing a target molecule complex using the hydrogel of any one of claim 1-30 or 36-44, the method comprising:
providing the hydrogel, wherein the hydrogel comprises a nucleic acid molecule, and the nucleic acid molecule comprises a cleavage moiety;
transcribing and translating the nucleic acid molecule to produce a plurality of target molecules; and
cleaving the cleavage moiety, thereby producing the target molecule complex.
